(19) United States
(12) Patent Application Publication
(10) Pub. No.: US 2010/0144764 A1
(43) Pub. Date: Jun. 10, 2010

(54) PYRIMIDINEDIONE DERIVATIVES AND METHODS OF USE THEREOF

(75) Inventors: Xianhai Huang, Warren, NJ (US); Anandan Palani, Bridgewater, NJ (US); Robert G. Aslanian, Rockaway, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/594,969

(22) PCT Filed: Apr. 9, 2008
(86) PCT No.: PCT/US08/04551

§ 371 Date: Dec. 10, 2009

Related U.S. Application Data
(60) Provisional application No. 60/911,679, filed on Apr. 13, 2007.

(51) Int. Cl. A61K 31/519; C07D 487/04; A61P 3/00; A61P 9/00; A61P 11/00; A61P 35/00; A61P 25/00; A61P 29/00; A61P 1/00; A61P 3/10

(52) U.S. Cl. 514/262.1; 544/256

(57) ABSTRACT

The present invention relates to Pyrimidinedione Derivatives, compositions comprising a Pyrimidinedione Derivative and methods for using the Pyrimidinedione Derivatives for treating or preventing a metabolic disorder, dyslipidemia, a cardiovascular disease, a neurological disorder, a hematological disease, cancer, inflammation, a respiratory disease, a gastroenterological disease, diabetes, a diabetic complication, obesity, an obesity-related disorder or non-alcoholic fatty liver disease.

### FIELD OF THE INVENTION

The present invention relates to Pyrimidinedione Derivatives, compositions comprising a Pyrimidinedione Derivative and methods for using the Pyrimidinedione Derivatives for treating or preventing a metabolic disorder, dyslipidemia, a cardiovascular disease, a neurological disorder, a hematological disease, cancer, inflammation, a respiratory disease, a gastroenterological disease, diabetes, a diabetic complication, obesity, an obesity-related disorder or non-alcoholic fatty liver disease.

### BACKGROUND OF THE INVENTION

Niacin, commonly known as nicotinic acid, plays an important role in the production of several sex and stress-related hormones, particularly those made by the adrenal gland. It also plays a role in removing toxic and harmful chemicals from the body.

When taken in large doses, nicotinic acid increases the level of high density lipoprotein (HDL) in blood, and is sometimes prescribed for patients with low HDL, and at high risk of heart attack. Nicotinic acid is also used in the treatment of hyperlipidemia because it reduces very low density lipoprotein (VLDL), a precursor of low density lipoprotein (LDL) secretion from the liver, and inhibits cholesterol synthesis. Nicotinic acid has also been used to treat metabolic syndrome, but there are problems with the clinical use of nicotinic acid, including skin flushing and diarrhea, even with moderate doses.

The use of heterocyclic compounds as nicotinic acid receptor agonists is known in the art and such compounds are disclosed, for example, in M. Ridi, Gazzetta Chim. Ital. (1950) vol. 80, p. 121 and M. Ridi, Gazzetta Chim. Ital. (1952) vol. 82, p. 23, which discloses syntheses of barbituric acid derivatives useful as nicotinic acid receptor (NAagonists. FR 2563223 discloses nucleoside analogs. T. Paterson et al., J. Chem. Soc., Perkins Trans. I (1972), vol. 8, pp. 1041-1050 discloses the synthesis of 8-substituted pyrido[2,3-d]pyrimidines. S. Rao, Indian J. Chem. (1974), 12(10), pp. 1028-1030 discloses the synthesis of pyrano[2,3-d]pyrimidines. M. Skof, Heterocycles, (1999), 51(5), pp. 1051-1058 discloses one step transformations of (S)-1-benzoyl-3-[(E)-dimethylaminomethylidene]-5-methoxycarbonyl-pyrrolidin-2-one into quinolizinyl- and 2H-2-pyranonyl-substituted alanine derivatives. R. Toplak J. Heterocyclic Chem. (1999), 36(1), pp. 225-235 discloses the synthesis of pyran-2-ones.

International Publication No. WO 04/110368 describes combination therapies for the treatment of hypertension comprising the combination of an anti-obesity agent and an anti-hypertensive agent.

International Publication No. WO 05/000217 describes combination therapies for the treatment of dyslipidemia comprising the administration of a combination of an anti-obesity agent and an anti-dyslipidemic agent.

International Publication No. WO 04/110375 describes combination therapies for the treatment of diabetes comprising the administration of a combination of an anti-obesity agent and an anti-diabetic agent.

U.S. Patent Publication No. 2004/0122033 describes combination therapies for the treatment of obesity comprising the administration of a combination of an appetite suppressant and/or metabolic rate enhancers and/or nutrient absorption inhibitors. U.S. Patent Publication No. 2004/0229844 describes combination therapies for treating atherosclerosis comprising the administration of a combination of nicotinic acid or another nicotinic acid receptor agonist and a DP receptor antagonist

International Publication No. WO05/077950 describes xanthine derivatives which are agonists of the nicotinic acid receptor HM74A.

Despite the medicinal chemistry efforts directed to discovering NAR receptor modulators, their remains a need in the art for NAR agonists with improved efficacy and reduced side effects. The present invention addresses this need.

### SUMMARY OF THE INVENTION

In one aspect, the present invention provides Compounds of Formula (I):

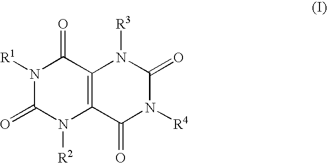

and pharmaceutically acceptable salts, solvates, esters and prodrugs thereof, wherein:

R¹ is H, alkyl, -(alkylene)ₙ-aryl, -(alkylene)ₙ-cycloalkyl, -(alkylene)ₙ-cycloalkenyl, -(alkylene)ₙheterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, heteroaryl, —OR⁵, —SR⁵, —N(R⁶)₂, —CN, —C(O)OR⁵ and —C(O)N(R⁶)₂;

R² is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵, —NHC(O)—R⁶ and —C(O)N(R⁶)₂;

R³ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R⁴ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

each occurrence of R⁵ is independently H, alkyl, aryl or cycloalkyl;

each occurrence of R⁶ is independently H, alkyl, -(alkylene)ₙ-aryl or cycloalkyl; and

each occurrence of n is independently 0 or 1.

In another aspect, the present invention provides Compounds of Formula (II):

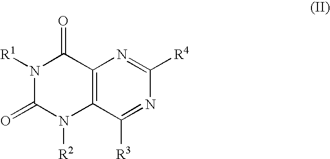

and pharmaceutically acceptable salts, solvates, esters and prodrugs thereof, wherein:

R¹ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R² is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R³ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R⁴ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

each occurrence of R⁵ is independently H, alkyl, aryl or cycloalkyl;

each occurrence of R⁶ is independently H, alkyl, -(alkylene)ₙ-aryl or cycloalkyl; and

each occurrence of n is independently 0 or 1.

In one aspect, the present invention provides Compounds of Formula (III):

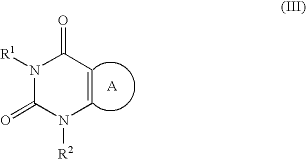

and pharmaceutically acceptable salts, solvates, esters and prodrugs thereof, wherein:

A is:

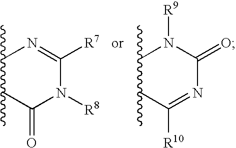

R¹ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, heteroaryl, —OR⁵, —SR⁵, —N(R⁶)₂, —CN, —C(O)OR⁵ and —C(O)N(R⁶)₂;

R² is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

each occurrence of R⁵ is independently H, alkyl, aryl or cycloalkyl;

each occurrence of R⁶ is independently H, alkyl, -(alkylene)ₙ-aryl or cycloalkyl;

R⁷ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —O-alkylene-O-aryl, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R⁸ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R⁹ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;

R¹⁰ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂; and

each occurrence of n is independently 0 or 1.

The compounds of formulas (I), (II) and (III) (the “Pyrimidinedione Derivatives”) are useful for treating or preventing a metabolic disorder, dyslipidemia, a cardiovascular disease, a neurological disorder, a hematological disease, cancer, inflammation, a respiratory disease, a gastroenterological disease, diabetes, a diabetic complicaton, obesity, an obesity-related disorder or non-alcoholic fatty liver disease (each being a “Condition”) in a patient.

In another aspect, the invention provides methods for treating a Condition in a patient, comprising administering to the patient an effective amount of one or more Pyrimidinedione Derivatives.

In a further aspect, the invention provides compositions comprising an effective amount of one or more Pyrimidinedione Derivatives and a pharmaceutically acceptable carrier.

### DETAILED DESCRIPTION OF THE INVENTION

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “impaired glucose tolerance” as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” as used herein, is defined as a fasting plasma glucose level of 100 to 125 mg/dL; normal fasting glucose values are below 100 mg per dL.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “obesity-related disorder” as used herein refers to: (i) disorders which result from a patient having a BMI of 25 or greater; and (ii) eating disorders and other disorders associated with excessive food intake. Non-limiting examples of an obesity-related disorder include edema, shortness of breath, sleep apnea, skin disorders and high blood pressure.

The term “metabolic syndrome” as used herein, refers to a set of risk factors that make a patient more susceptible to cardiovascular disease and/or type 2 diabetes. A patient is said to have metabolic syndrome if the patient simultaneously has three or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “effective amount” as used herein, refers to an amount of a Pyrimidinedione Derivative and/or an additional therapeutic agent, or a composition thereof that is effective in producing the desired therapeutic, ameliorative, inhibitory or preventative effect when administered to a patient suffering from a Condition. When more than one Pyrimidinedione Derivative is present, or in the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkyl, aryl, cycloalkyl, cyano, alkoxy and —S(alkyl). In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of alkyl, aryl and cycloalkyl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. In one embodiment, an alkylene group has from 1 to about 6 carbon atoms. In another embodiment, an alkylene group is branched. In another embodiment, an alkylene group is linear.

“Aryl” means an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 5 to about 7 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “cycloalkenyl,” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is joined via a ring carbon atom and any nitrogen atom of a heteroaryl can be optionally oxidized to the corresponding N-oxide. The term “heteroaryl” also encompasses a heteroaryl group, as defined above, which has been fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(Boc), —N(Cbz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocyclyl can be optionally oxidized to the corresponding N-oxide, S-oxide or S,S-dioxide. Non-limiting examples of monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is pyrrolidonyl:

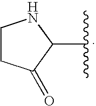

The term “heterocycloalkenyl,” as used herein, refers to a heterocycloalkyl group, as defined above, wherein the heterocycloalkyl group contains from 3 to 10 ring atoms, and at least one endocyclic carbon-carbon or carbon-nitrogen double bond. In one embodiment, a heterocycloalkenyl group has from 5 to 10 ring atoms. In another embodiment, a heterocycloalkenyl group is monocyclic and has 5 or 6 ring atoms. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. A ring carbon atom of a heterocycloalkyl group may be functionalized as a carbonyl group. An illustrative example of such a heterocycloalkenyl group is:

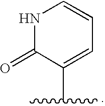

The term “5-membered heterocycloalkenyl,” as used herein, refers to a heterocycloalkenyl group, as defined above, which has 5 ring atoms.

It should also be noted that tautomeric forms such as, for example, the moieties:

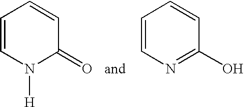

are considered equivalent in certain embodiments of this invention.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkyl-aryl, -aryl-alkyl, -alkylene-heteroaryl, -alkenylene-heteroaryl, -alkynylene-heteroaryl, hydroxy, hydroxyalkyl, haloalkyl, —O-alkyl, -alkylene-O-alkyl, —O-aryl, aralkoxy, acyl, aroyl, halo, nitro, cyano, carboxy, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, cycloalkyl, and alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

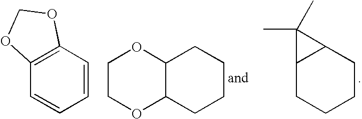

“Halo” means —F, —Cl, —Br or —I. In one embodiment, halo refers to —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., R¹, R², n, etc. . . . ) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein.

A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a Pyrimidinedione Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Pyrimidinedione Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Pyrimidinedione Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a Pyrimidinedione Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of Solvates is Generally Known. Thus, for Example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Pyrimidinedione Derivatives can form salts which are also within the scope of this invention. Reference to a Pyrimidinedione Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Pyrimidinedione Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of the Formula (I) may be formed, for example, by reacting a Pyrimidinedione Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Pyrimidinedione Derivatives may be atropisomers (e.g., substituted biaryls) and are considered as part of this invention. Enantiomers can also be separated by use of chiral HPLC column.

It is also possible that the Pyrimidinedione Derivatives may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Pyrimidinedione Derivative incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Pyrimidinedione Derivatives (e.g., those labeled with ³H and ¹⁴C) are useful in compound and/or substrate tissue distribution assays. In one embodiment, ritiated (i.e., ³H) and carbon-14 (i.e., ¹⁴C) isotopes are employed for their ease of preparation and detectability. In another embodiment, substitution with heavier isotopes such as deuterium (i.e., ²H) may afford certain therapeutic advantages resulting from greater metabolic stability (e.g., increased in vivo half-life or reduced dosage requirements).

Isotopically labelled Pyrimidinedione Derivatives

Synthetic chemical procedures analogous to those disclosed herein for making the Pyrimidinedione Derivatives, by substituting an appropriate isotopically labelled starting material or reagent for a non-isotopically labelled starting material or reagent.

Polymorphic forms of the Pyrimidinedione Derivatives, and of the salts, solvates, hydrates, esters and prodrugs of the Pyrimidinedione Derivatives, are intended to be included in the present invention.

The following abbreviations are used herein and have the following meanings: n-Bu is n-butyl, CDI is 1,1′-carbonyldiimidazole, dba is dibenzylideneacetone, DMF is N,N-dimethylformamide, DMSO is dimethylsulfoxide, EtOAc is ethyl acetate, EtOH is ethanol, HOAc is acetic acid, HPLC is high performance liquid chromatography, Me is methyl, MS is N-iodosuccinimide, PBS is phosphate-buffered saline, Ph is phenyl, PPh₃ is triphenylphoshpine and TFAA is trifluoroacetic acid.

### The Compounds of Formula (I)

The present invention provides compounds having the formula (I):

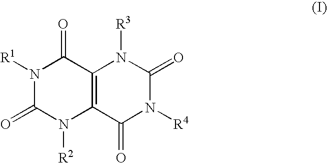

and pharmaceutically acceptable salts, solvates, esters and prodrugs thereof, wherein R¹, R², R³ and R⁴ are defined above for the compounds of formula (I).

In one embodiment, R¹ is H.

In another embodiment, R¹ is alkyl.

In another embodiment, R¹ is aryl.

In still another embodiment, R¹ is cycloalkyl.

In yet another embodiment, R¹ is cycloalkenyl.

In another embodiment, R¹ is heterocycloalkyl.

In a further embodiment, R¹ is heterocycloalkenyl.

In another embodiment, R¹ is heteroaryl.

In one embodiment, R¹ is -(alkylene)-aryl.

In another embodiment, R¹ is -(alkylene)-cycloalkyl.

In another embodiment, R¹ is -(alkylene)-cycloalkenyl.

In yet another embodiment, R¹ is -(alkylene)-heterocycloalkyl.

In another embodiment, R¹ is (alkylene)-heterocycloalkenyl.

In a further embodiment, R¹ is -(alkylene)-heteroaryl.

In one embodiment, R¹ is methyl.

In another embodiment, R¹ is n-butyl.

In another embodiment, R¹ is:

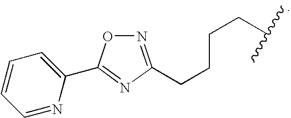

In one embodiment, R² is H.

In another embodiment, R² is alkyl.

In another embodiment, R² is aryl.

In still another embodiment, R² is cycloalkyl.

In yet another embodiment, R² is cycloalkenyl.

In another embodiment, R² is heterocycloalkyl.

In a further embodiment, R² is heterocycloalkenyl.

In another embodiment, R² is heteroaryl.

In one embodiment, R² is -(alkylene)-aryl.

In another embodiment, R² is -(alkylene)-cycloalkyl.

In another embodiment, R² is -(alkylene)-cycloalkenyl.

In yet another embodiment, R² is -(alkylene)-heterocycloalkyl.

In another embodiment, R² is (alkylene)-heterocycloalkenyl.

In a further embodiment, R² is -(alkylene)-heteroaryl.

In one embodiment, R² is H.

In another embodiment, R² is methyl.

In another embodiment, R² is ethyl.

In still another embodiment, R² is n-propyl.

In yet another embodiment, R² is n-butyl.

In another embodiment, R² is n-pentyl.

In a further embodiment, R² is n-hexyl.

In one embodiment, R³ is H.

In another embodiment, R³ is alkyl.

In another embodiment, R³ is aryl.

In still another embodiment, R³ is cycloalkyl.

In yet another embodiment, R³ is cycloalkenyl.

In another embodiment, R³ is heterocycloalkyl.

In a further embodiment, R³ is heterocycloalkenyl.

In another embodiment, R³ is heteroaryl.

In one embodiment, R³ is -(alkylene)-aryl.

In another embodiment, R³ is -(alkylene)-cycloalkyl.

In another embodiment, R³ is -(alkylene)-cycloalkenyl.

In yet another embodiment, R³ is -(alkylene)-heterocycloalkyl.

In another embodiment, R³ is (alkylene)-heterocycloalkenyl.

In a further embodiment, R³ is -(alkylene)-heteroaryl.

In another embodiment, R³ is —OR⁵.

In another embodiment, R³ is —N(R⁶)₂.

In one embodiment, R³ is H.

In another embodiment, R³ is methyl.

In one embodiment, R⁴ is H.

In another embodiment, R⁴ is alkyl.

In another embodiment, R⁴ is aryl.

In still another embodiment, R⁴ is cycloalkyl.

In yet another embodiment, R⁴ is cycloalkenyl.

In another embodiment, R⁴ is heterocycloalkyl.

In a further embodiment, R⁴ is heterocycloalkenyl.

In another embodiment, R⁴ is heteroaryl.

In one embodiment, R⁴ is -(alkylene)-aryl.

In another embodiment, R⁴ is -(alkylene)-cycloalkyl.

In another embodiment, R⁴ is -(alkylene)-cycloalkenyl.

In yet another embodiment, R⁴ is -(alkylene)-heterocycloalkyl.

In another embodiment, R⁴ is (alkylene)-heterocycloalkenyl.

In a further embodiment, R⁴ is -(alkylene)-heteroaryl.

In another embodiment, R⁴ is —OR⁵.

In another embodiment, R⁴ is —N(R⁶)₂.

In one embodiment, R⁴ is H.

In another embodiment, R⁴ is methyl.

In one embodiment, R¹ is H and R² is alkyl.

In another embodiment, R¹ is H and R² is n-butyl.

In one embodiment, R¹ and R² are each alkyl.

In another embodiment, R¹ is methyl and R² is alkyl.

In another embodiment, R¹ is methyl and R² is n-butyl.

In still another embodiment, R¹ and R² are the same.

In yet another embodiment, R¹ and R² are different.

In a further embodiment, R¹ and R² are each n-pentyl.

In one embodiment, R³ is H and R⁴ is alkyl.

In another embodiment, R³ is alkyl and R⁴ is H.

In another embodiment, R³ and R⁴ are each H.

In still another embodiment, R³ and R⁴ are the same.

In yet another embodiment, R³ and R⁴ are different.

In a further embodiment, R¹ and R² are each alkyl.

In one embodiment, R′, R³ and R⁴ are each H.

In another embodiment, R′, R³ and R⁴ are each H and R² is alkyl.

In another embodiment, R¹ and R² are each alkyl and R³ and R⁴ are each H.

In still another embodiment, R¹ is H and R², R³ and R⁴ are each alkyl.

In yet another embodiment, R¹ and R³ are each H and R² and R⁴ are each alkyl.

In a further embodiment, R¹ and R⁴ are each H and R² and R³ are each alkyl.

Non-limiting examples of compounds of formula (I) include the following compounds:

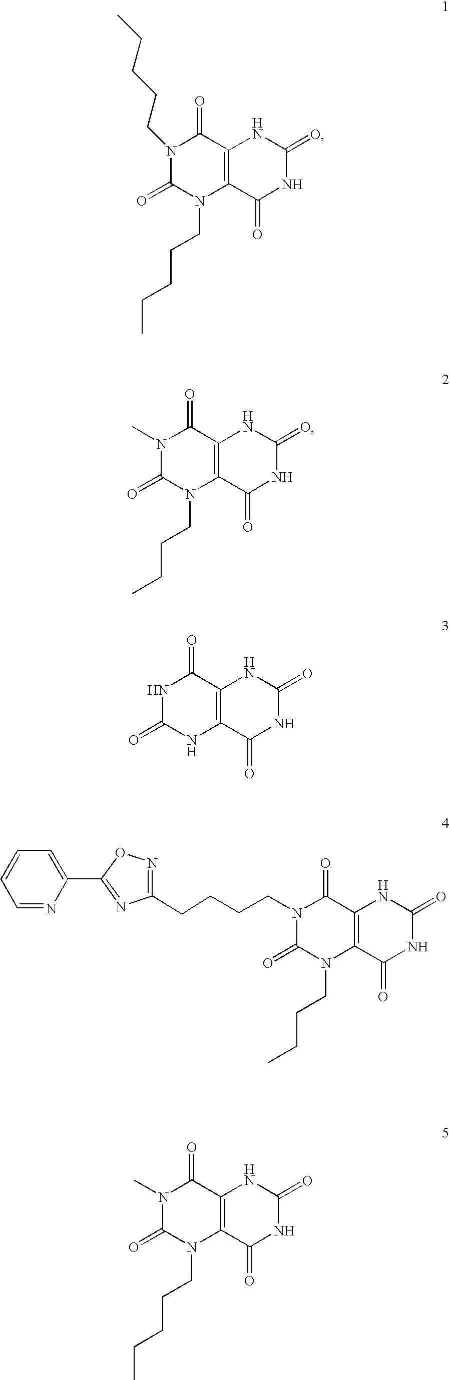

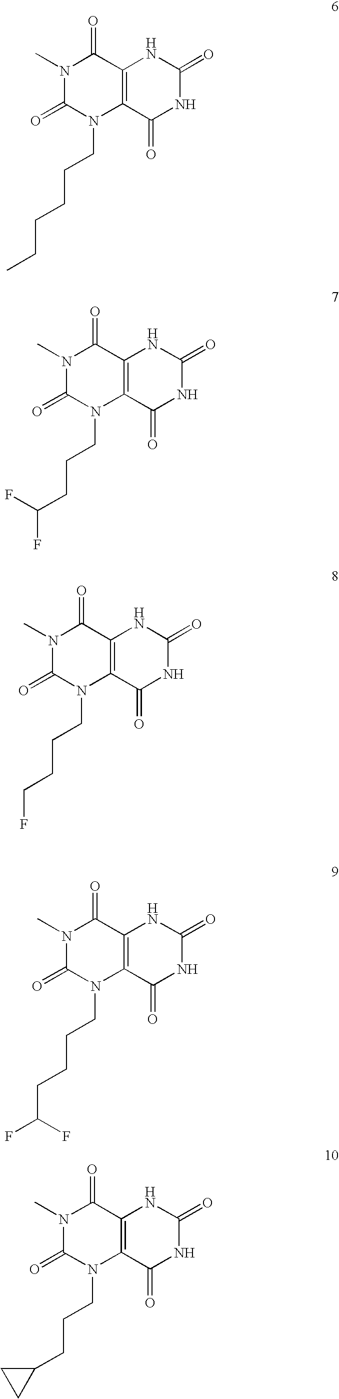

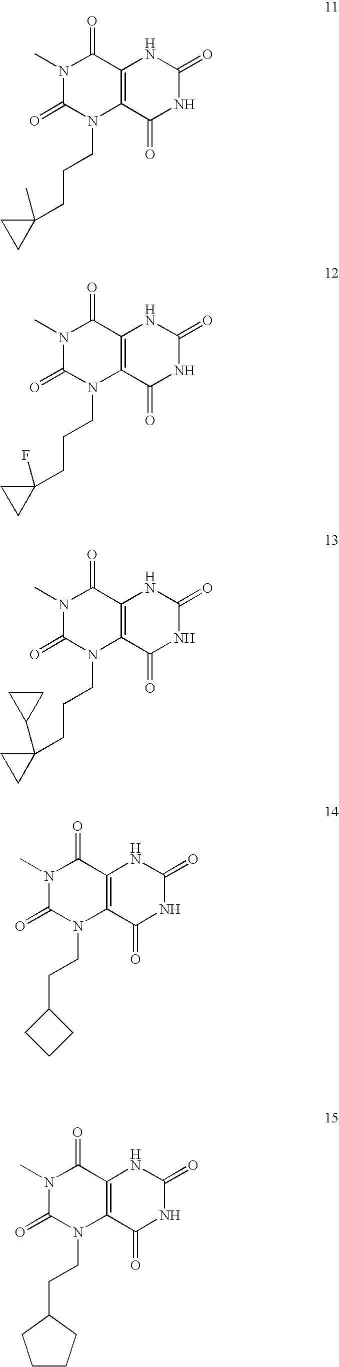

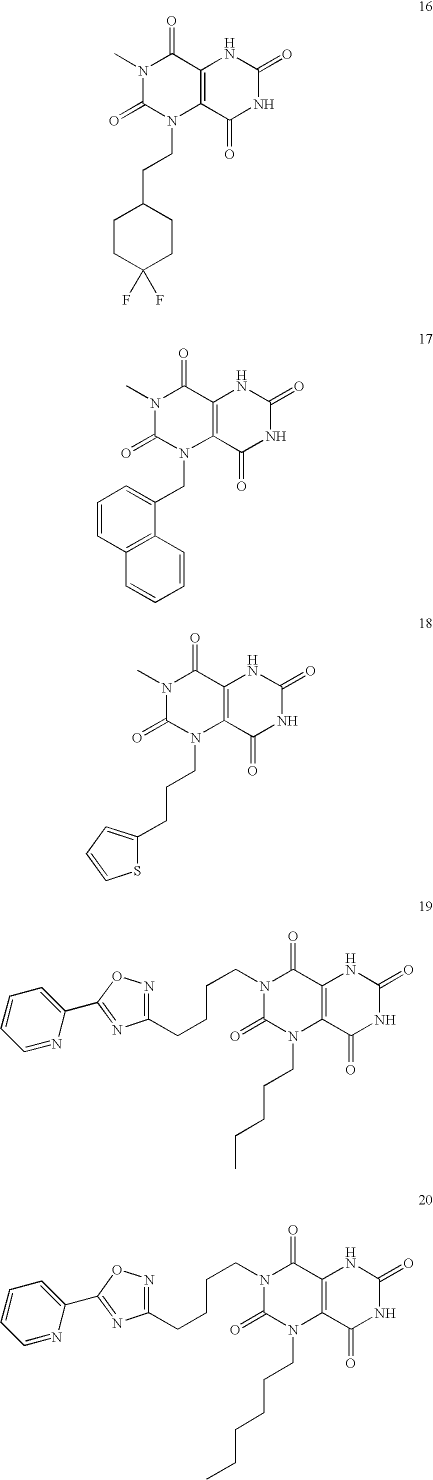

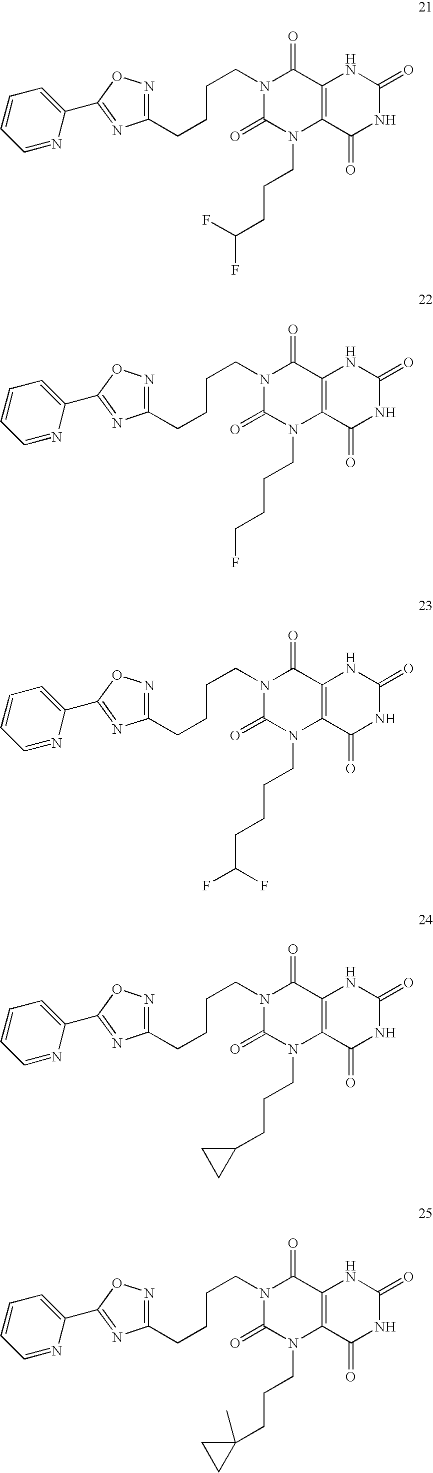

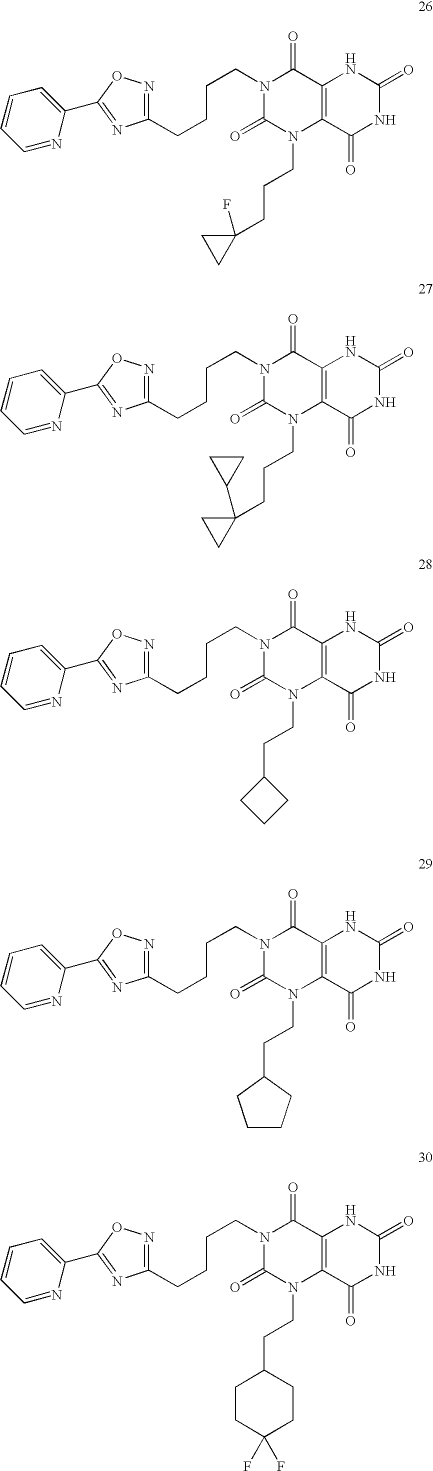

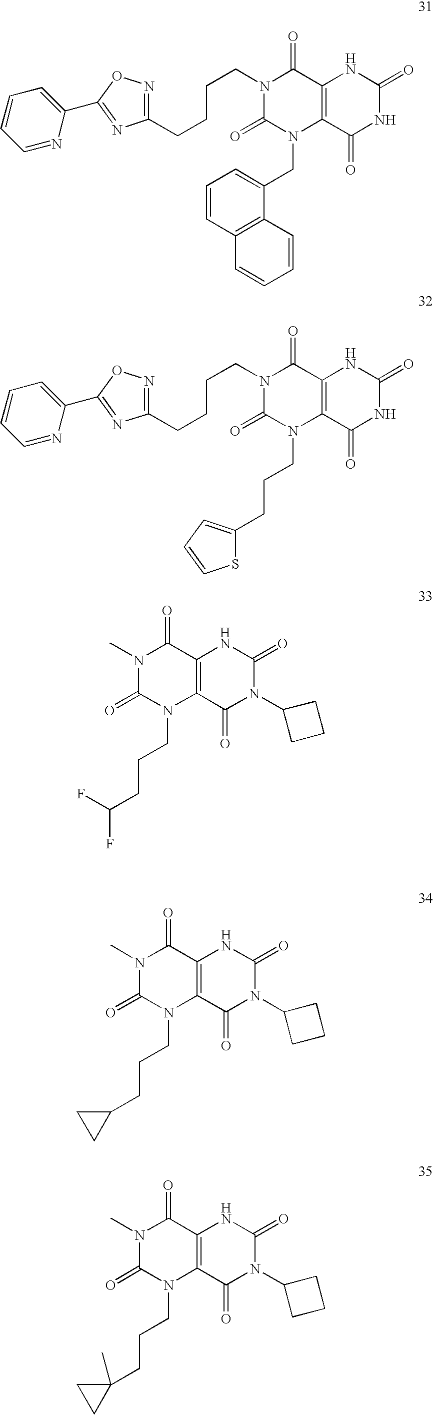

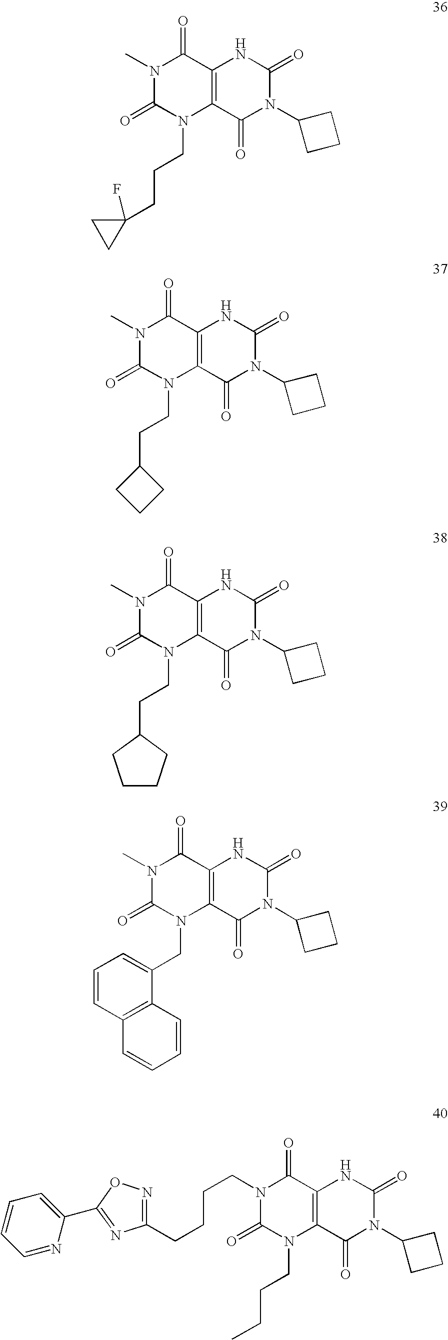

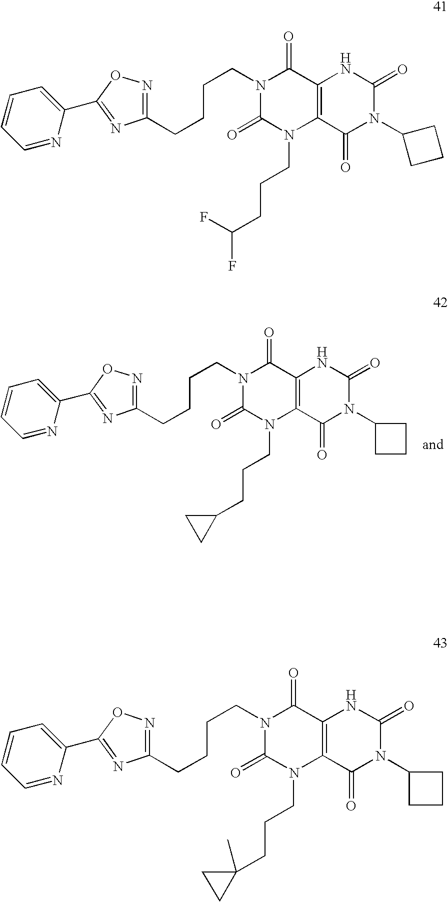

and pharmaceutically acceptable salts, solvates, esters and prodrugs thereof.

### The Compounds of Formula (II)

The present invention provides compounds having the formula (II):

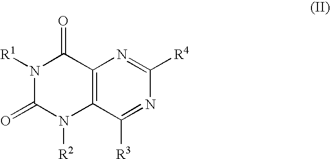

and pharmaceutically acceptable salts, solvates, esters and prodrugs thereof, wherein R¹, R², R³ and R⁴ are defined above for the compounds of formula (II).

In one embodiment, R¹ is H.

In another embodiment, R¹ is alkyl.

In another embodiment, R¹ is aryl.

In still another embodiment, R¹ is cycloalkyl.

In yet another embodiment, R¹ is cycloalkenyl.

In another embodiment, R¹ is heterocycloalkyl.

In a further embodiment, R¹ is heterocycloalkenyl.

In another embodiment, R¹ is heteroaryl.

In one embodiment, R¹ is -(alkylene)-aryl.

In another embodiment, R¹ is -(alkylene)-cycloalkyl.

In another embodiment, R¹ is -(alkylene)-cycloalkenyl.

In yet another embodiment, R¹ is -(alkylene)-heterocycloalkyl.

In another embodiment, R¹ is (alkylene)-heterocycloalkenyl.

In a further embodiment, R¹ is -(alkylene)-heteroaryl.

In one embodiment, R¹ is methyl.

In another embodiment, R¹ is n-butyl.

In one embodiment, R² is H.

In another embodiment, R² is alkyl.

In another embodiment, R² is aryl.

In still another embodiment, R² is cycloalkyl.

In yet another embodiment, R² is cycloalkenyl.

In another embodiment, R² is heterocycloalkyl.

In a further embodiment, R² is heterocycloalkenyl.

In another embodiment, R² is heteroaryl.

In one embodiment, R² is -(alkylene)-aryl.

In another embodiment, R² is -(alkylene)-cycloalkyl.

In another embodiment, R² is -(alkylene)-cycloalkenyl.

In yet another embodiment, R² is -(alkylene)-heterocycloalkyl.

In another embodiment, R² is (alkylene)-heterocycloalkenyl.

In a further embodiment, R² is -(alkylene)-heteroaryl.

In one embodiment, R² is H.

In another embodiment, R² is methyl.

In another embodiment, R² is ethyl.

In still another embodiment, R² is n-propyl.

In yet another embodiment, R² is n-butyl.

In another embodiment, R² is n-pentyl.

In a further embodiment, R² is n-hexyl.

In one embodiment, R³ is H.

In another embodiment, R³ is alkyl.

In another embodiment, R³ is haloalkyl.

In another embodiment, R³ is aryl.

In still another embodiment, R³ is cycloalkyl.

In yet another embodiment, R³ is cycloalkenyl.

In another embodiment, R³ is heterocycloalkyl.

In a further embodiment, R³ is heterocycloalkenyl.

In another embodiment, R³ is heteroaryl.

In one embodiment, R³ is -(alkylene)-aryl.

In another embodiment, R³ is -(alkylene)-cycloalkyl.

In another embodiment, R³ is -(alkylene)-cycloalkenyl.

In yet another embodiment, R³ is -(alkylene)-heterocycloalkyl.

In another embodiment, R³ is (alkylene)-heterocycloalkenyl.

In a further embodiment, R³ is -(alkylene)-heteroaryl.

In another embodiment, R³ is —OR⁵.

In another embodiment, R³ is —N(R⁶)₂.

In still another embodiment, R³ is —SR⁵.

In yet another embodiment, R³ is —CN.

In another embodiment, R³ is —C(O)OR⁵.

In a further embodiment, R³ is —C(O)N(R⁶)₂.

In one embodiment, R³ is H.

In another embodiment, R³ is methyl.

In one embodiment, R⁴ is H.

In another embodiment, R⁴ is alkyl.

In another embodiment, R⁴ is haloalkyl.

In another embodiment, R⁴ is aryl.

In still another embodiment, R⁴ is cycloalkyl.

In yet another embodiment, R⁴ is cycloalkenyl.

In another embodiment, R⁴ is heterocycloalkyl.

In a further embodiment, R⁴ is heterocycloalkenyl.

In another embodiment, R⁴ is heteroaryl.

In one embodiment, R⁴ is -(alkylene)-aryl.

In another embodiment, R⁴ is -(alkylene)-cycloalkyl.

In another embodiment, R⁴ is -(alkylene)-cycloalkenyl.

In yet another embodiment, R⁴ is -(alkylene)-heterocycloalkyl.

In another embodiment, R⁴ is (alkylene)-heterocycloalkenyl.

In a further embodiment, R⁴ is -(alkylene)-heteroaryl.

In another embodiment, R⁴ is —OR⁵.

In another embodiment, R⁴ is —N(R⁶)₂.

In still another embodiment, R⁴ is —SR⁵.

In yet another embodiment, R⁴ is —CN.

In another embodiment, R⁴ is —C(O)OR⁵.

In a further embodiment, R⁴ is —C(O)N(R⁶)₂.

In one embodiment, R⁴ is H.

In another embodiment, R⁴ is methyl.

In one embodiment, R¹ is H and R² is alkyl.

In another embodiment, R¹ is H and R² is n-butyl.

In one embodiment, R¹ and R² are each alkyl.

In another embodiment, R¹ is methyl and R² is alkyl.

In another embodiment, R¹ is methyl and R² is n-butyl.

In still another embodiment, R¹ and R² are the same.

In yet another embodiment, R¹ and R² are different.

In a further embodiment, R¹ and R² are each n-pentyl.

In one embodiment, R³ is H and R⁴ is alkyl.

In another embodiment, R³ is alkyl and R⁴ is H.

In another embodiment, R³ and R⁴ are each H.

In still another embodiment, R³ and R⁴ are the same.

In yet another embodiment, R³ and R⁴ are different.

In a further embodiment, R¹ and R² are each alkyl.

In one embodiment, R¹, R³ and R⁴ are each H.

In another embodiment, R¹, R³ and R⁴ are each H and R² is alkyl.

In another embodiment, R¹ and R² are each alkyl and R³ and R⁴ are each H.

In still another embodiment, R¹ is H and R², R³ and R⁴ are each alkyl.

In yet another embodiment, R¹ and R³ are each H and R² and R⁴ are each alkyl.

In a further embodiment, R¹ and R⁴ are each H and R² and R³ are each alkyl.

Non-limiting examples of compounds of formula (II) include the following compounds:

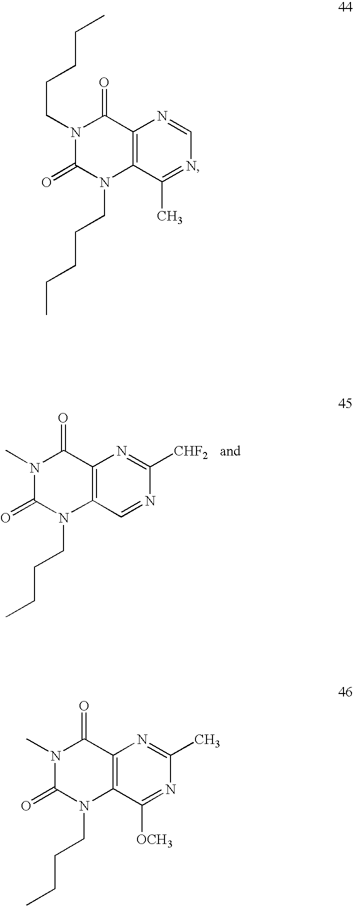

and pharmaceutically acceptable salts, solvates, esters and prodrugs thereof.

### The Compounds of Formula (III)

The present invention provides compounds having the formula (III):

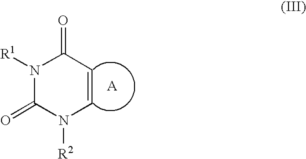

and pharmaceutically acceptable salts, solvates, esters and prodrugs thereof, wherein: A, R¹, R², R⁷, R⁸, R⁹ and R¹⁰ are defined above for the compounds of formula (III).

In one embodiment, A is:

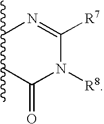

In another embodiment, A is:

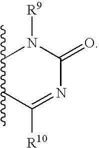

In one embodiment, R¹ is H.

In another embodiment, R¹ is alkyl.

In another embodiment, R¹ is aryl.

In still another embodiment, R¹ is cycloalkyl.

In yet another embodiment, R¹ is cycloalkenyl.

In another embodiment, R¹ is heterocycloalkyl.

In a further embodiment, R¹ is heterocycloalkenyl.

In another embodiment, R¹ is heteroaryl.

In one embodiment, R¹ is -(alkylene)-aryl.

In another embodiment, R¹ is -(alkylene)-cycloalkyl.

In another embodiment, R¹ is -(alkylene)-cycloalkenyl.

In yet another embodiment, R¹ is -(alkylene)-heterocycloalkyl.

In another embodiment, R¹ is (alkylene)-heterocycloalkenyl.

In a further embodiment, R¹ is -(alkylene)-heteroaryl.

In one embodiment, R¹ is methyl.

In another embodiment, R¹ is n-butyl.

In another embodiment, R¹ is:

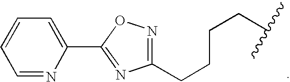

In one embodiment, R² is H.

In another embodiment, R² is alkyl.

In another embodiment, R² is aryl.

In still another embodiment, R² is cycloalkyl.

In yet another embodiment, R² is cycloalkenyl.

In another embodiment, R² is heterocycloalkyl.

In a further embodiment, R² is heterocycloalkenyl.

In another embodiment, R² is heteroaryl.

In one embodiment, R² is -(alkylene)-aryl.

In another embodiment, R² is -(alkylene)-cycloalkyl.

In another embodiment, R² is -(alkylene)-cycloalkenyl.

In yet another embodiment, R² is -(alkylene)-heterocycloalkyl.

In another embodiment, R² is (alkylene)-heterocycloalkenyl.

In a further embodiment, R² is -(alkylene)-heteroaryl.

In one embodiment, R² is H.

In another embodiment, R² is methyl.

In another embodiment, R² is ethyl.

In still another embodiment, R² is n-propyl.

In yet another embodiment, R² is n-butyl.

In another embodiment, R² is n-pentyl.

In a further embodiment, R² is n-hexyl.

In one embodiment, R⁷ is H.

In another embodiment, R⁷ is alkyl.

In another embodiment, R⁷ is haloalkyl.

In another embodiment, R⁷ is aryl.

In still another embodiment, R⁷ is cycloalkyl.

In yet another embodiment, R⁷ is cycloalkenyl.

In another embodiment, R⁷ is heterocycloalkyl.

In a further embodiment, R⁷ is heterocycloalkenyl.

In another embodiment, R⁷ is heteroaryl.

In one embodiment, R⁷ is -(alkylene)-aryl.

In another embodiment, R⁷ is -(alkylene)-cycloalkyl.

In another embodiment, R⁷ is -(alkylene)-cycloalkenyl.

In yet another embodiment, R⁷ is -(alkylene)-heterocycloalkyl.

In another embodiment, R⁷ is (alkylene)-heterocycloalkenyl. In a further embodiment, R⁷ is -(alkylene)-heteroaryl.

In another embodiment, R⁷ is —OR⁵.

In another embodiment, R⁷ is —N(R⁶)₂.

In still another embodiment, R⁷ is —SR⁵.

In yet another embodiment, R⁷ is —CN.

In another embodiment, R⁷ is —C(O)OR⁵.

In a further embodiment, R⁷ is —C(O)N(R⁶)₂.

In one embodiment, R⁷ is H.

In another embodiment, R⁷ is methyl.

In still another embodiment, R⁷ is —O-alkyl.

In yet another embodiment, R⁷ is methoxy.

In another embodiment, R⁷ is —CHF₂.

In another embodiment, R⁷ is:

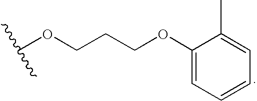

In one embodiment, R⁸ is H.

In another embodiment, R⁸ is alkyl.

In another embodiment, R⁸ is aryl.

In still another embodiment, R⁸ is cycloalkyl.

In yet another embodiment, R⁸ is cycloalkenyl.

In another embodiment, R⁸ is heterocycloalkyl.

In a further embodiment, R⁸ is heterocycloalkenyl.

In another embodiment, R⁸ is heteroaryl.

In one embodiment, R⁸ is -(alkylene)-aryl.

In another embodiment, R⁸ is -(alkylene)-cycloalkyl.

In another embodiment, R⁸ is -(alkylene)-cycloalkenyl.

In yet another embodiment, R⁸ is -(alkylene)-heterocycloalkyl.

In another embodiment, R⁸ is (alkylene)-heterocycloalkenyl.

In a further embodiment, R⁸ is -(alkylene)-heteroaryl.

In another embodiment, R⁸ is —OR⁵.

In another embodiment, R⁸ is —N(R⁶)₂.

In one embodiment, R⁸ is H.

In another embodiment, R⁸ is methyl.

In one embodiment, R⁹ is H.

In another embodiment, R⁹ is alkyl.

In another embodiment, R⁹ is aryl.

In still another embodiment, R⁹ is cycloalkyl.

In yet another embodiment, R⁹ is cycloalkenyl.

In another embodiment, R⁹ is heterocycloalkyl.

In a further embodiment, R⁹ is heterocycloalkenyl.

In another embodiment, R⁹ is heteroaryl.

In one embodiment, R⁹ is -(alkylene)-aryl.

In another embodiment, R⁹ is -(alkylene)-cycloalkyl.

In another embodiment, R⁹ is -(alkylene)-cycloalkenyl.

In yet another embodiment, R⁹ is -(alkylene)-heterocycloalkyl.

In another embodiment, R⁹ is (alkylene)-heterocycloalkenyl.

In a further embodiment, R⁹ is -(alkylene)-heteroaryl.

In another embodiment, R⁹ is —OR⁵.

In another embodiment, R⁹ is —N(R⁶)₂.

In one embodiment, R⁹ is H.

In another embodiment, R⁹ is methyl.

In one embodiment, R¹⁰ is H.

In another embodiment, R¹⁰ is alkyl.

In another embodiment, R¹⁰ is haloalkyl.

In another embodiment, R¹⁰ is aryl.

In still another embodiment, R¹⁰ is cycloalkyl.

In yet another embodiment, R¹⁰ is cycloalkenyl.

In another embodiment, R¹⁰ is heterocycloalkyl.

In a further embodiment, R¹⁰ is heterocycloalkenyl.

In another embodiment, R¹⁰ is heteroaryl.

In one embodiment, R¹⁰ is -(alkylene)-aryl.

In another embodiment, R¹⁰ is -(alkylene)-cycloalkyl.

In another embodiment, R¹⁰ is -(alkylene)-cycloalkenyl.

In yet another embodiment, R¹⁰ is -(alkylene)-heterocycloalkyl.

In another embodiment, R¹⁰ is (alkylene)-heterocycloalkenyl.

In a further embodiment, R¹⁰ is -(alkylene)-heteroaryl.

In another embodiment, R¹⁰ is —OR⁵.

In another embodiment, R¹⁰ is —N(R⁶)₂.

In still another embodiment, R¹⁰ is —SR⁵.

In yet another embodiment, R¹⁰ is —CN.

In another embodiment, R¹⁰ is —C(O)OR⁵.

In a further embodiment, R¹⁰ is —C(O)N(R⁶)₂.

In one embodiment, R¹⁰ is H.

In another embodiment, R¹⁰ is methyl.

In still another embodiment, R¹⁰ is ±0-alkyl.

In yet another embodiment, R¹⁰ is methoxy.

In another embodiment, R¹⁰ is —CHF₂.

In one embodiment, R¹ is:

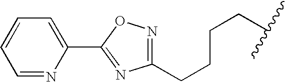

and

R⁷ is:

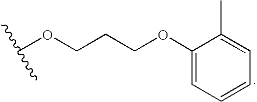

In one embodiment, A is:

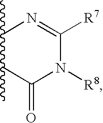

one of R¹ and R² is H and the other is alkyl.

In another embodiment, A is:

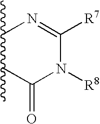

and R¹ and R² are each alkyl.

In another embodiment, A is:

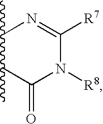

R¹ and R² are each alkyl, and R⁷ is haloalkyl.

In another embodiment, A is:

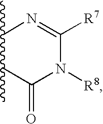

R¹ and R² are each alkyl, and R⁷ is —CHF₂.

In still another embodiment, A is:

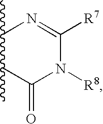

R¹ and R² are each alkyl, and R⁷ is alkyl.

In still another embodiment, A is:

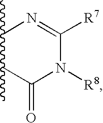

R¹ and R² are each alkyl, and R⁷ is —O-alkyl.

In still another embodiment, A is:

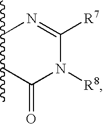

R¹ and R² are each alkyl, and R⁷ is —OCH₃.

In yet another embodiment, A is:

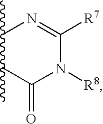

R¹ and R² are each alkyl, and R⁸ is H.

In a further embodiment, A is:

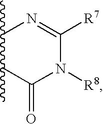

R¹ and R² are each alkyl, and R⁸ is alkyl.

In one embodiment, A is:

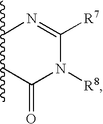

R¹ and R² are each alkyl, R⁷ is alkyl and R⁸ is H.

In another embodiment, A is:

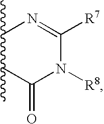

R¹ and R² are each alkyl, R⁷ is —O-alkyl and R⁸ is H.

In another embodiment, A is:

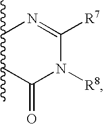

R¹ and R² are each alkyl, R⁷ is methoxy and R⁸ is H.

In a further embodiment, A is:

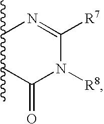

R¹ and R² are each alkyl, R⁷ is haloalkyl and R⁸ is H.

In another embodiment, A is:

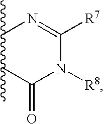

R¹ and R² are each alkyl, R⁷ is —CHF₂ and R⁸ is H.

In another embodiment, A is:

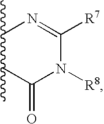

R¹ and R² are each alkyl, R⁷ is alkyl and R⁸ is alkyl.

In another embodiment, A is:

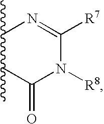

R¹ and R² are each alkyl, R⁷ is haloalkyl and R⁸ is alkyl.

In a further embodiment, A is:

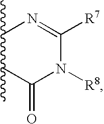

R¹ and R² are each alkyl, R⁷ is —CHF₂ and R⁸ is alkyl.

In one embodiment, A is:

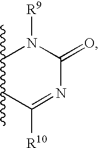

one of R¹ and R² is H and the other is alkyl.

In another embodiment, A is:

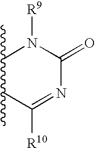

and R¹ and R² are each alkyl.

In another embodiment, A is:

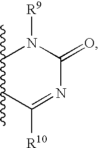

R¹ and R² are each alkyl, and R¹⁰ is haloalkyl.

In yet another embodiment, A is:

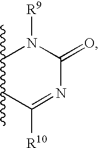

R¹ and R² are each alkyl, and R¹⁰ is —CHF₂.

In still another embodiment, A is:

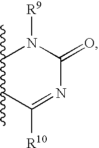

R¹ and R² are each alkyl, and R¹⁰ is alkyl.

In another embodiment, A is:

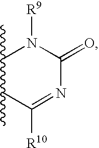

R¹ and R² are each alkyl, and R¹⁰ is —O-alkyl.

In one embodiment, A is:

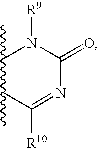

R¹ and R² are each alkyl, and R¹⁰ is methoxy.

In yet another embodiment, A is:

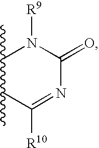

R¹ and R² are each alkyl, and R⁹ is H.

In a further embodiment, A is:

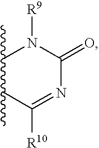

R¹ and R² are each alkyl, and R⁹ is alkyl.

In one embodiment, A is:

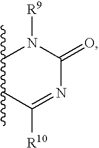

R¹ and R² are each alkyl, R⁹ is H and R¹⁰ is alkyl.

In another embodiment, A is:

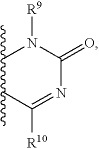

R¹ and R² are each alkyl, R⁹ is H and R¹⁰ is —O-alkyl.

In another embodiment, A is:

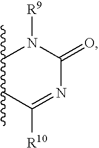

R¹ and R² are each alkyl, R⁹ is H and R¹⁰ is methoxy.

In a further embodiment, A is:

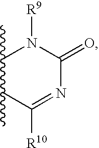

R¹ and R² are each alkyl, R⁹ is H and R¹⁰ is haloalkyl.

In one embodiment, A is:

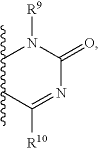

R¹ and R² are each alkyl, R⁹ is H and R¹⁰ is —CHF₂.

In another embodiment, A is:

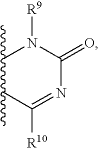

R¹ and R² are each alkyl, R⁹ is alkyl and R¹⁰ is alkyl.

In still another embodiment, A is:

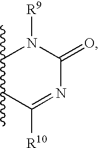

R¹ and R² are each alkyl, R⁹ is alkyl and R¹⁰ is —O-alkyl.

In yet another embodiment, A is:

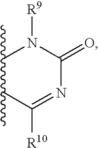

R¹ and R² are each alkyl, R⁹ is alkyl and R¹⁰ is methoxy.

In another embodiment, A is:

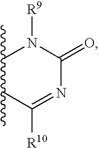

R¹ and R² are each alkyl, R⁹ is alkyl and R¹⁰ is haloalkyl.

In another embodiment, A is:

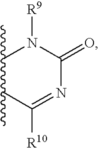

R¹ and R² are each alkyl, R⁹ is alkyl and R¹⁰ is —CHF₂.

Non-limiting examples of compounds of formula (III) include the following compounds:

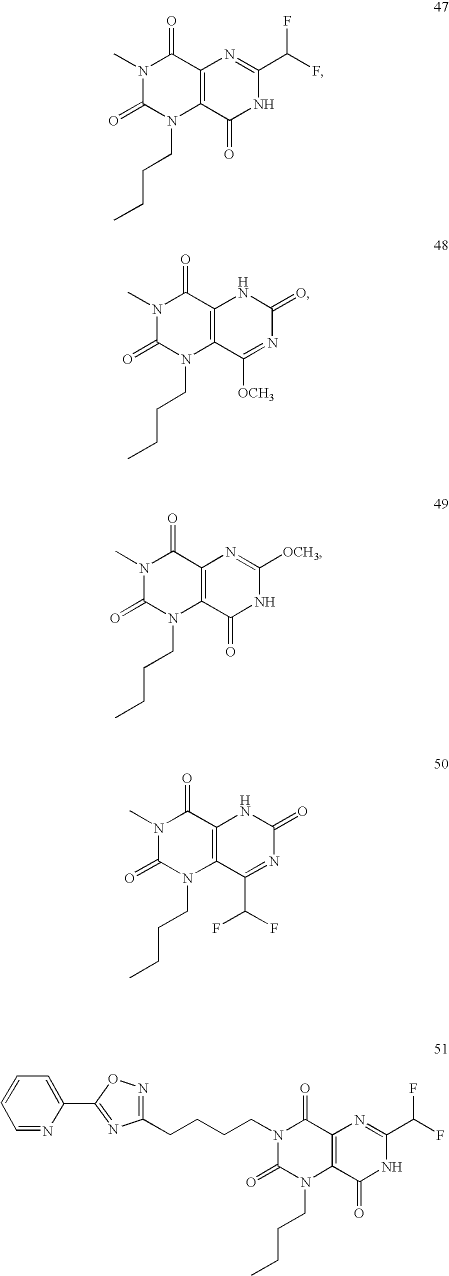

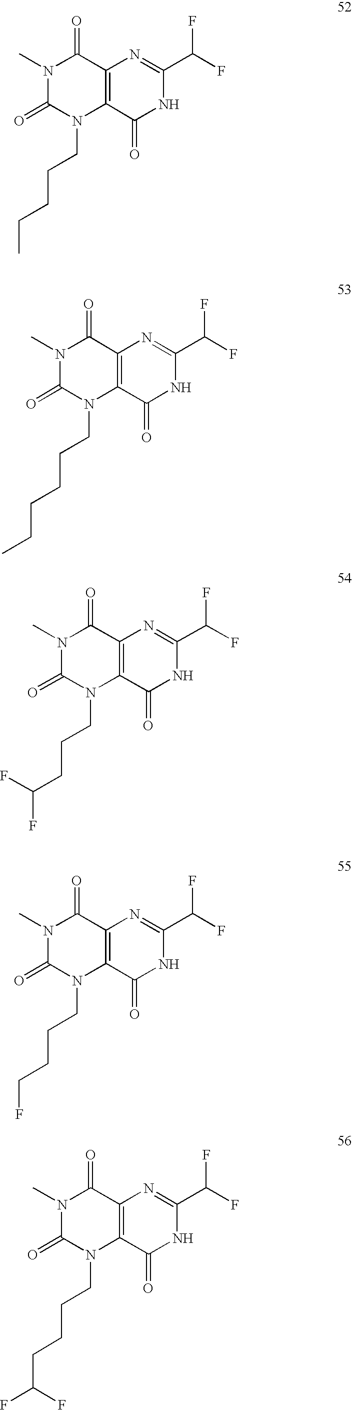

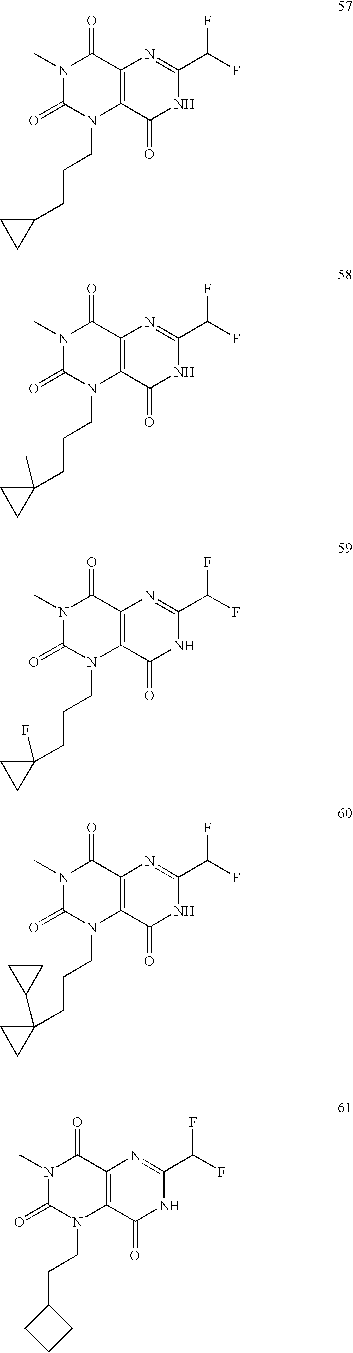

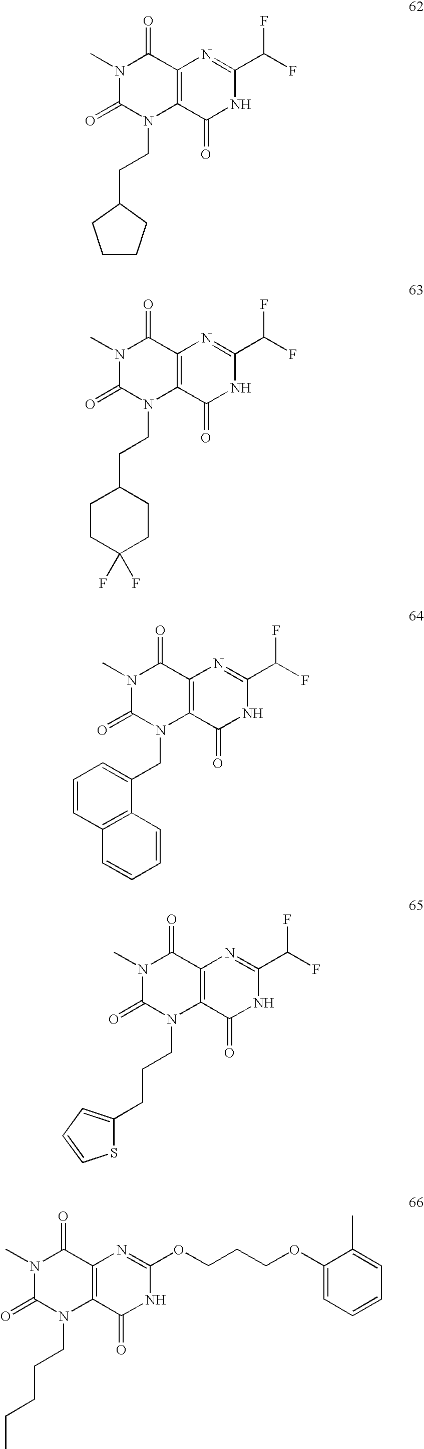

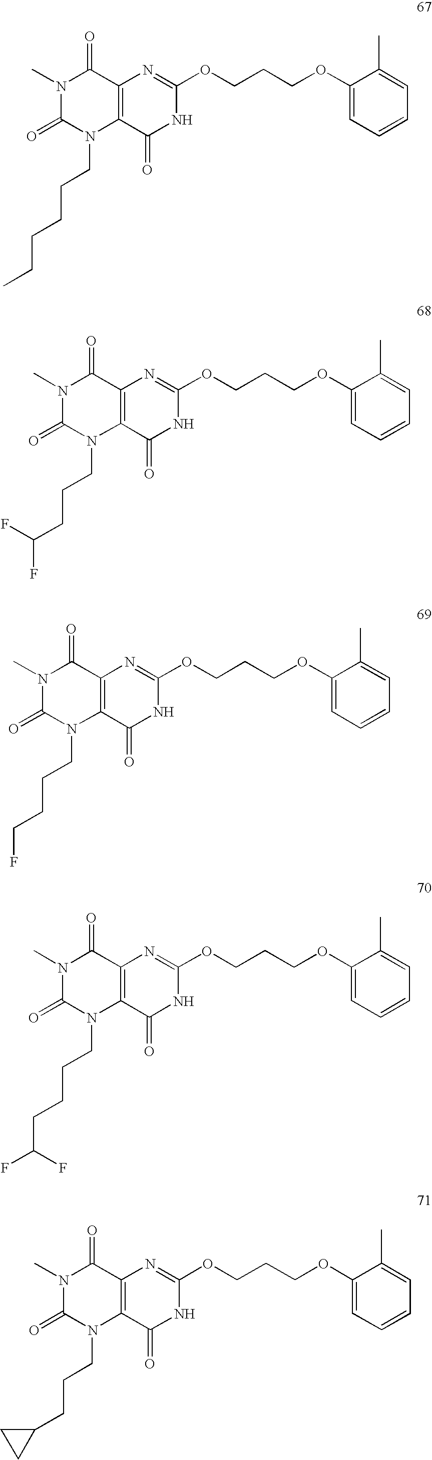

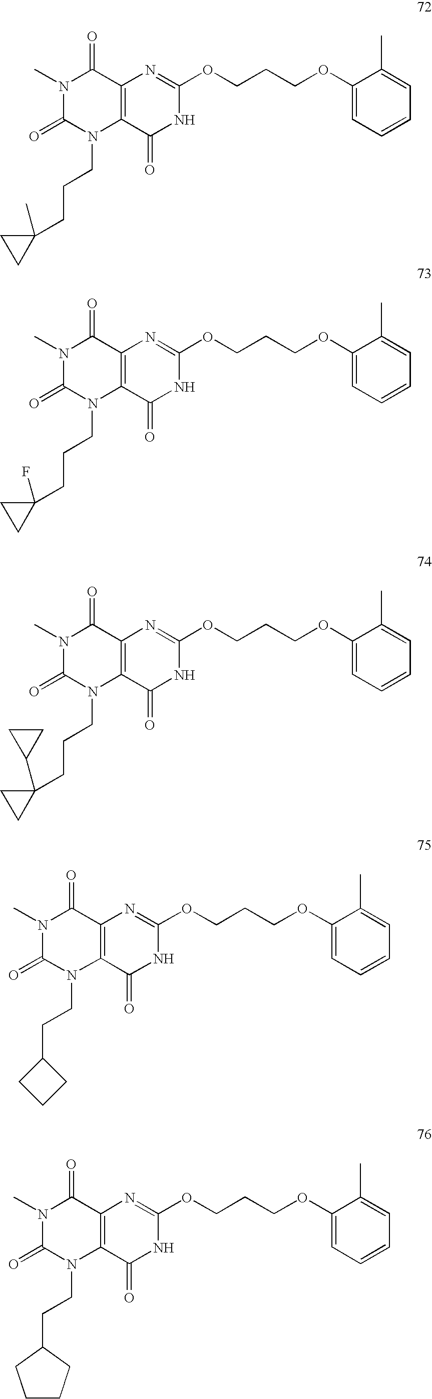

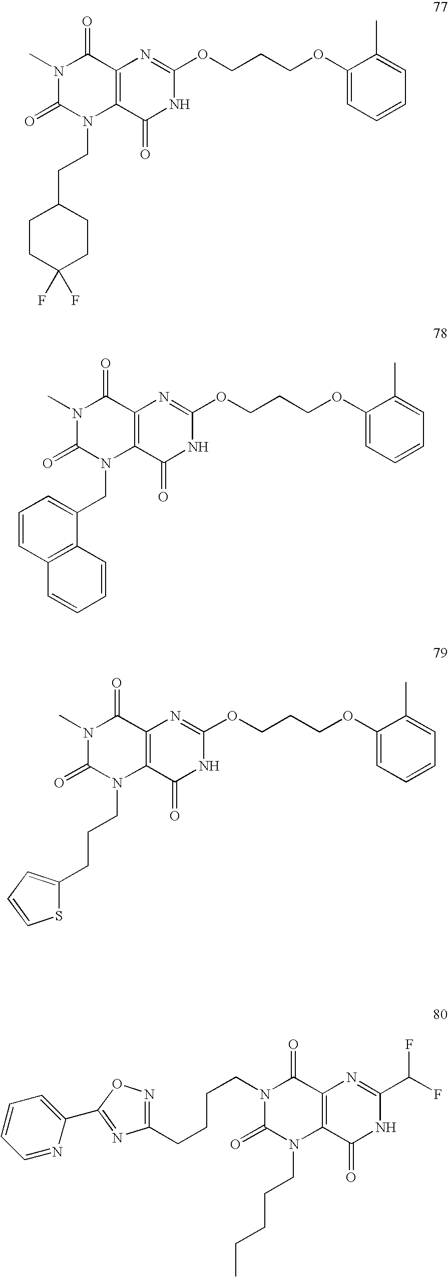

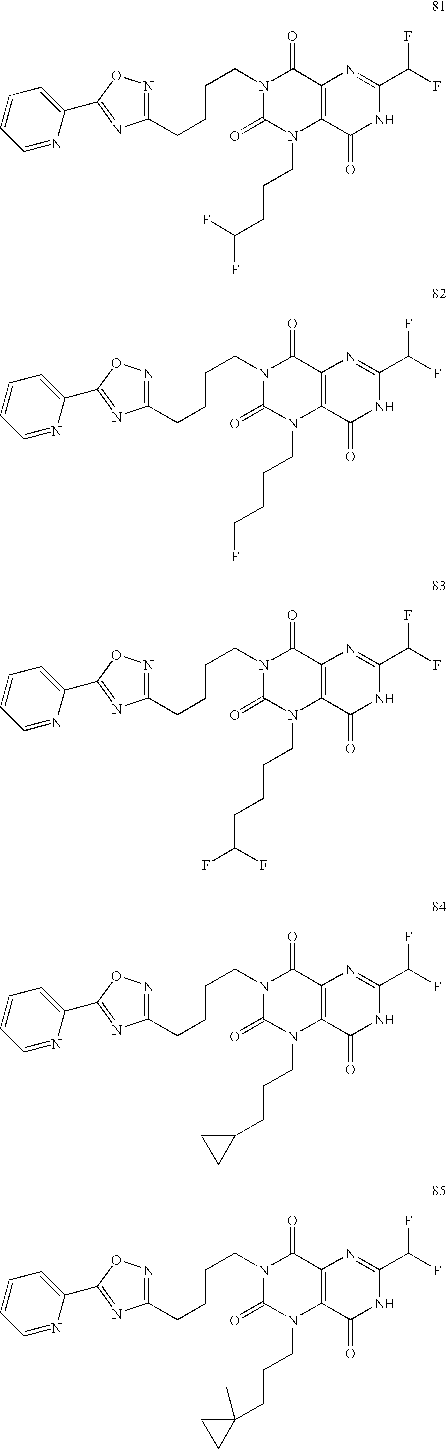

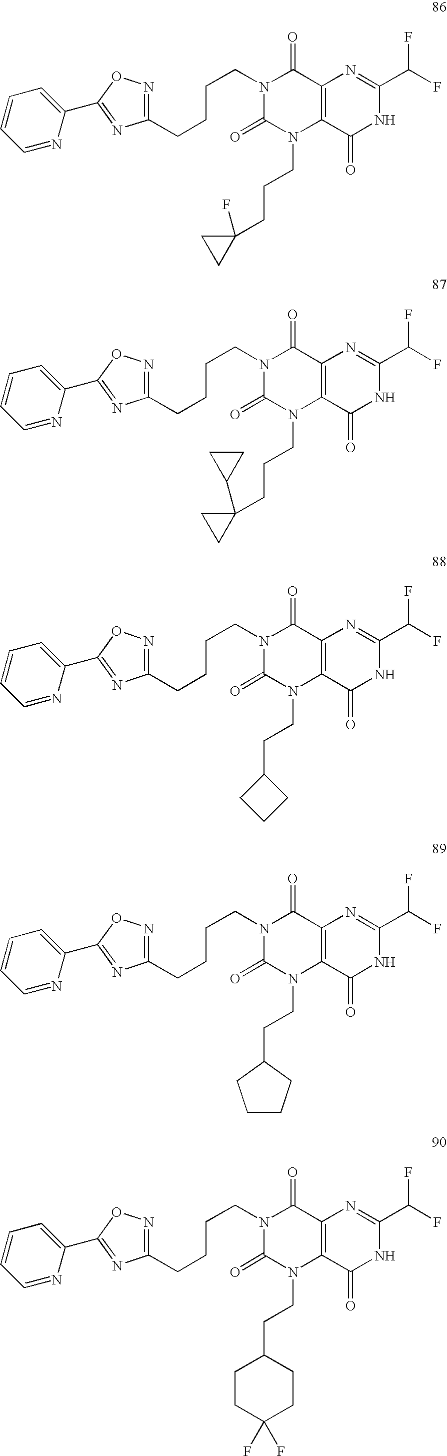

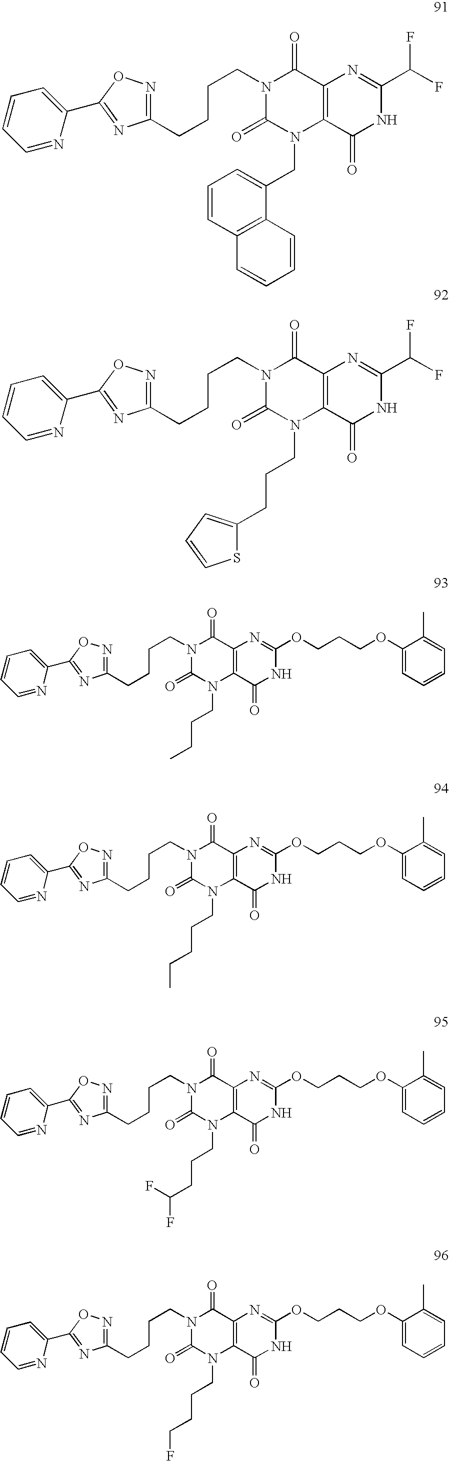

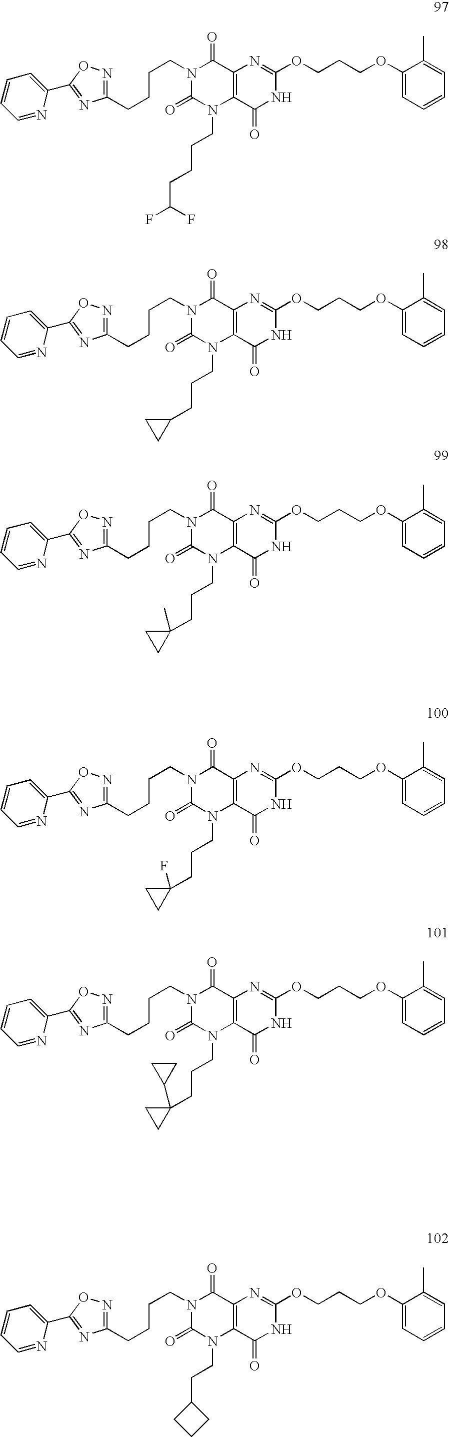

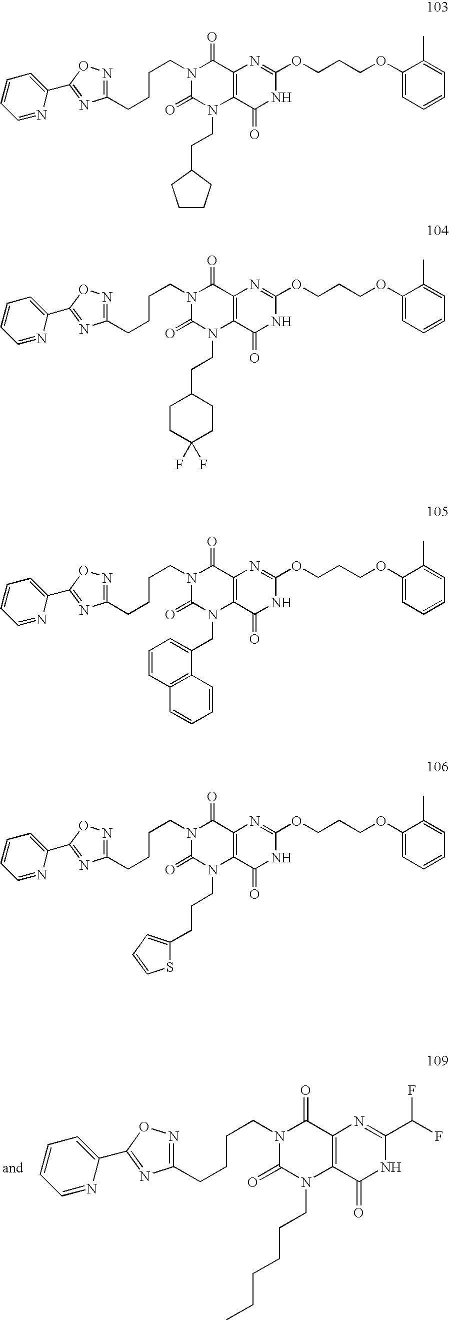

and pharmaceutically acceptable salts, solvates, esters and prodrugs thereof.

### Methods for Making the Pyrimidinedione Derivatives

Methods useful for making the Pyrimidinedione Derivatives are set forth below in Schemes 1-8. Alternative synthetic pathways and analogous structures will be apparent to those skilled in the art or organic synthesis.

Scheme 1 illustrates a method for making the Pyrimidinedione Derivatives of formula (I), wherein R³ and R⁴ are each H.

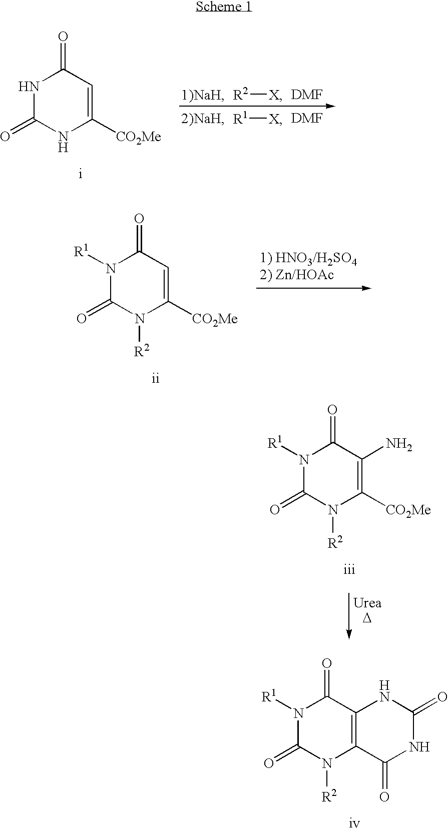

A compound of formula i can be alkylated at both amino groups using alkylating agents R¹—X or R²—X in the presence sodium hydride to provide the ester compounds of formula u. A compound of formula ii can then be nitrated using a mixture of nitric and sulfuric acid, followed by subsequent reduction of the nitro group using zinc-acetic acid reduction provides the corresponding amino compounds of formula iii. The compounds of formula iii can then be reacted with urea at elevated temperature to provide the compounds of formula iv, which correspond to the Pyrimidinedione Derivatives of formula (I), wherein R³ and R⁴ are each H. Further elaboration of the compounds of formula iv to provide the compounds of formula (I) wherein R³ and/or R⁴ are other than H can be accomplished using methods known to one skilled in the art of organic synthesis, including certain methods disclosed herein.

Scheme 2 illustrates a method for making the Pyrimidinedione Derivatives of formulas (I) and (III).

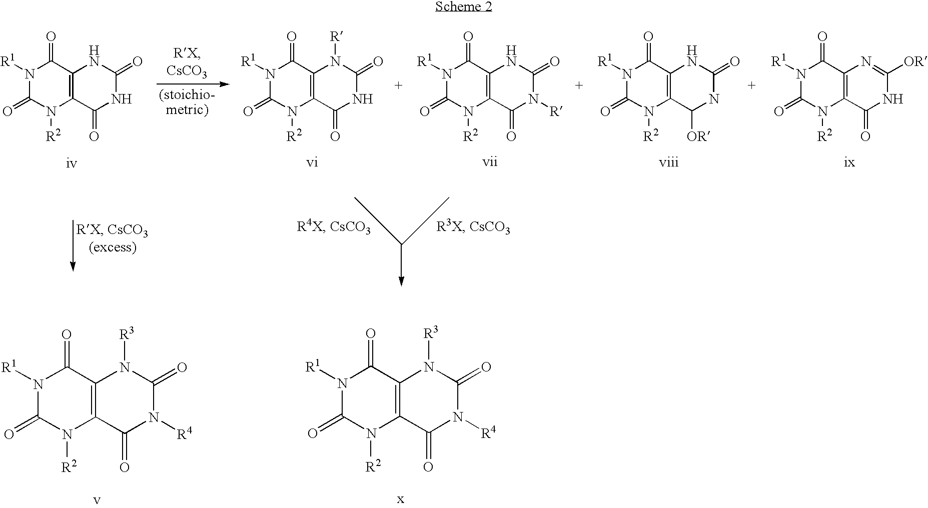

A bicyclic compound of formula iv can be reacted with an excess of compound of formula R′X in the presence of a non-nucleophilic base, such as cesium carbonate, to provide the dialkylated compounds of formula v, which correspond to the Pyrimidinedione Derivatives of formula (I), wherein R³ and R⁴ are the same and correspond to R′.

Alternatively, a bicyclic compound of formula iv can be reacted with a stoichiometric amount of a compound of formula R′X in the presence of a non-nucleophilic base, such as cesium carbonate, to provide a mixture of compounds of formula vi (which correspond to the Pyrimidinedione Derivatives of formula (I), wherein R⁴ is H and R³ is other than H), formula vu (which correspond to the Pyrimidinedione Derivatives of formula (I), wherein R³ is H and R⁴ is other than H), formula viii (which correspond to the Pyrimidinedione Derivatives of formula (III), wherein R⁹ is H and R¹⁰ is —OR⁵) and formula ix (which correspond to the Pyrimidinedione Derivatives of formula (I), wherein R⁷ is —OR⁵ and R⁸ is H) which can be separated and isolated using well-known separation techniques. The compounds of formula vi can be further reacted with a compound of formula R⁴X in the presence of a non-nucleophilic base, such as cesium carbonate, to provide the compounds of formula x, which correspond to the Pyrimidinedione Derivatives of formula (I), wherein R³ and R⁴ are different and are each other than H. The compounds of formula vii can be further reacted with a compound of formula R³X using the same method to provide the Pyrimidinedione Derivatives of formula (I), wherein R³ and R⁴ are different and are each other than H.

Scheme 3 illustrates a method for making the Pyrimidinedione Derivatives of formula (II), wherein R⁷ is —CHF₂ and R⁸ is H.

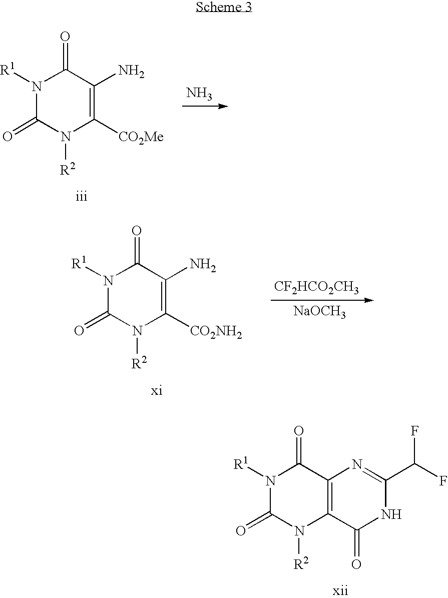

A dione compound of formula iii can be reacted with ammonia to provide the amido compounds of formula xi. The amido compounds of formula xi can subsequently be reacted with methyldifluoroacetate to provide the bicyclic compounds of formula xii, which correspond to the Pyrimidinedione Derivatives of formula (III), wherein R⁷ is —CHF₂ and R⁸ is H.

Scheme 4 illustrates a method for making the Pyrimidinedione Derivatives of formula (II), wherein R³ is —Cl, alkyl, aryl, heteroaryl or H and R⁴ is defined above for the compounds of formula (II).

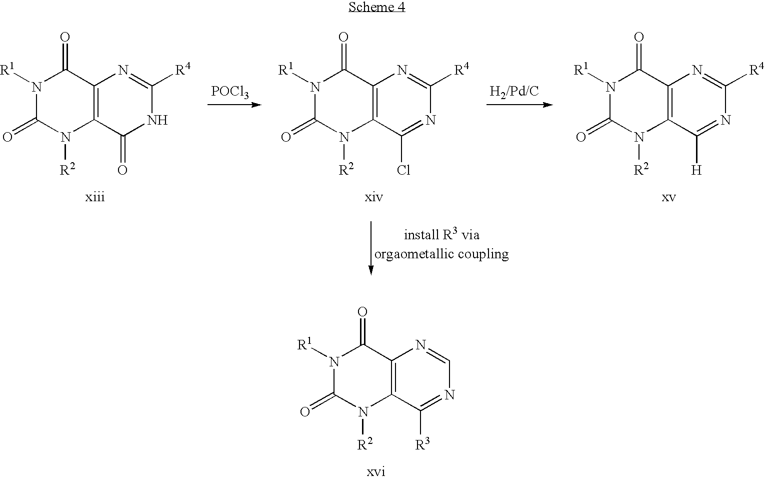

wherein R¹, R² and R⁴ are defined above for the compounds of formula (II) and R³ represents any R³ group, as defined above for the compounds of formula (II), which can be installed using organometallic coupling methodology with the aryl chloride compounds of formula xiv.

A compound of formula xiii can be reacted with phosphorus oxychloride to provide the compounds of formula xiv, which correspond to the Pyrimidinedione Derivatives of formula (II), wherein R³ is —Cl. The compounds of formula xiv can then be hydrogenated using Pd/C, for example, to provide the compounds of formula xv, which correspond to the Pyrimidinedione Derivatives of formula (II), wherein R³ is —H. It will be apparent to one skilled in the art of organic synthesis how to apply this methodology to obtain the analogous the Pyrimidinedione Derivatives of formula (II), wherein R³ is —Cl or H and R⁴ is defined above for the compounds of formula (II). The compounds of formula xiv can be further elaborated using organometallic coupling methods, such as a Suzuki Coupling, a Stille Coupling, a Kumada coupling, etc. . . . , to provide compounds of formula xvi, which correspond to the Pyrimidinedione Derivatives of formula (II), wherein R³ is alkyl, aryl or heteroaryl. The compounds of formula xiv can be further elaborated beyond what is shown above using methods known to one skilled in the art of organic synthesis to provide other compounds within the scope of formula (II).

Scheme 5 illustrates a method for making the Pyrimidinedione Derivatives of formula (II), wherein R³ is —OR⁵ and R⁴ is defined above for the compounds of formula (II).

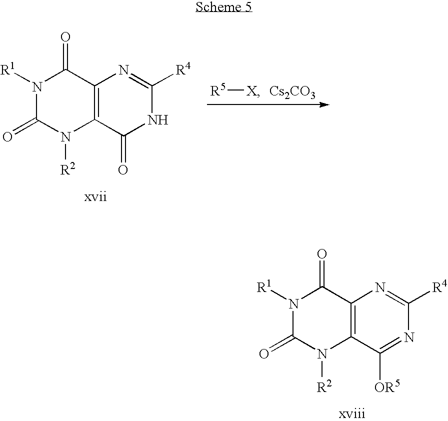

wherein R¹, R², R⁴ and R⁵ are defined above for the compounds of formula (II) and X is a good leaving group, such as —Cl, —Br, —I, —O-tosyl, —O-mesyl or —O-triflate.

A compound of formula xvii can be reacted with a compound of formula R⁵—X in the presence of a non-nucleophilic base, such as cesium carbonate, to provide the compounds of formula xviii, which correspond to the Pyrimidinedione Derivatives of formula (II), wherein R³ is —OR⁵ and R⁴ is defined above for the compounds of formula (II). It will be apparent to one skilled in the art of organic synthesis how to apply this methodology to obtain the analogous the Pyrimidinedione Derivatives of formula (II), wherein R⁴ is —OR⁵ and R³ is defined above for the compounds of formula (II).

Scheme 6 illustrates a method for making the Pyrimidinedione Derivatives of formula (III), wherein R¹ is:

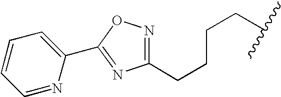

### and A is:

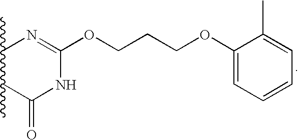

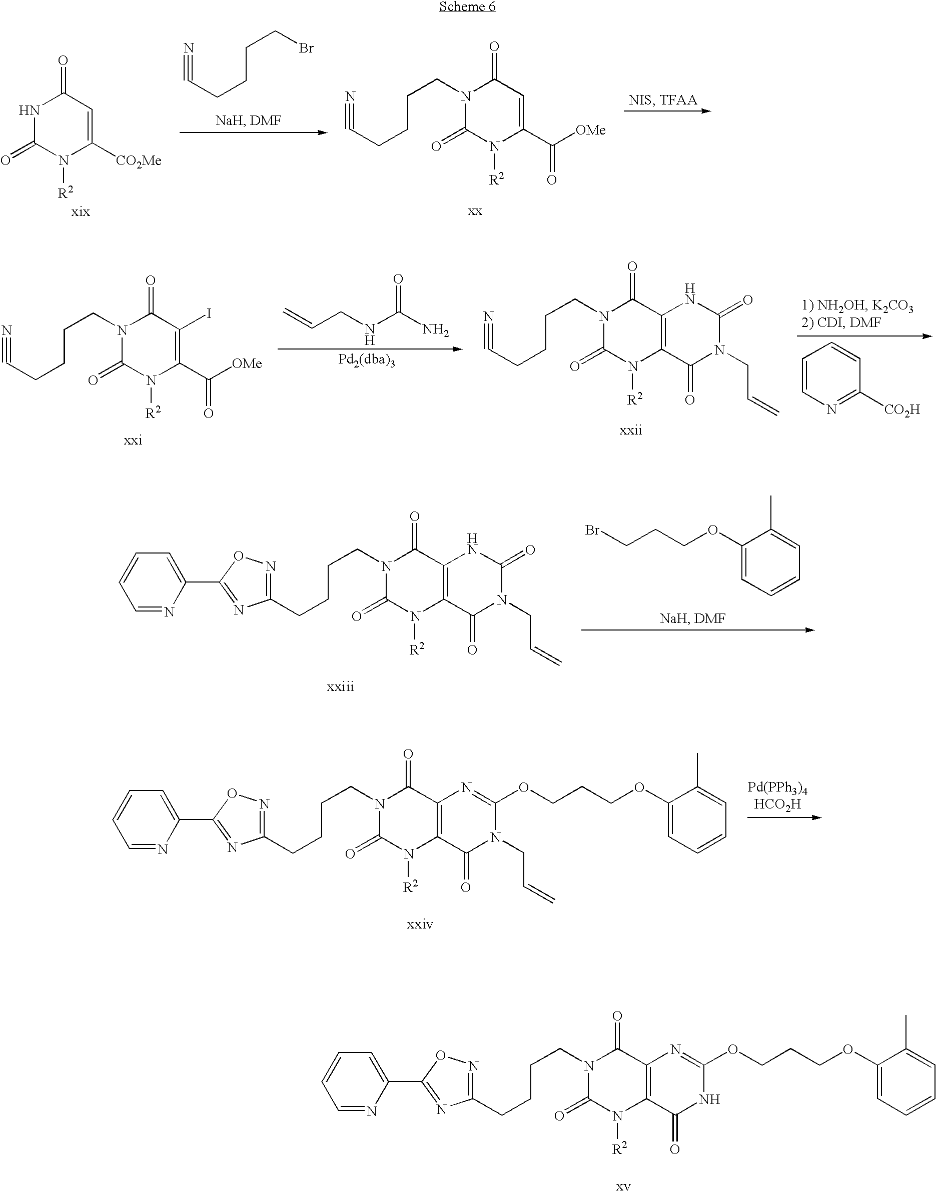

wherein R¹ and R² are defined above for the compounds of formula (II) and X is a good leaving group, such as —Cl, —Br, —I, —O-tosyl, —O-mesyl or —O-triflate.

An ester of formula xix (which can be made using the method described above in the first step of scheme 1) can be reacted with in the presence of sodium hydride to provide the compounds of formula xx. A compound of formula xx can then be iodinated using N-iodosuccinimide in the presence of trifluoroacetic acid to provide the iodo intermediates of formula xxi. A palladium catalyzed coupling of a compound of formula xxi provides the bicyclic allyl compounds of formula xxii. Reacton of the nitrile group of the compounds of formula xxii with 2-pyridine carboxylic acid in the presence of hydroxylamine and potassium carbonate, followed by reaction of the resulting adduct with CDI, provides the compounds of formula xxiii, which has the desired R¹ group intact. A compound of formula xxiii can then further derivatized via reaction with in the presence of sodium hydride to provide the compounds of formula xxiv, which can be treated with tetrakistriphenylphosphine palladium (IV) in the presence of formic acid to remove the allyl protecting group and provide the compounds of formula xv, which correspond to the compounds of formula (III) wherein R¹ is:

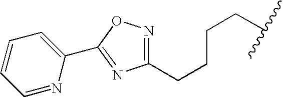

### and A is:

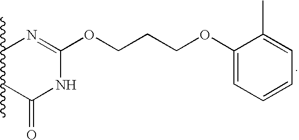

Scheme 7 illustrates a method for making the Pyrimidinedione Derivatives of formula (I), wherein R¹ is:

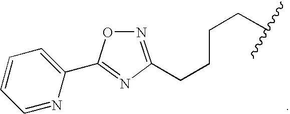

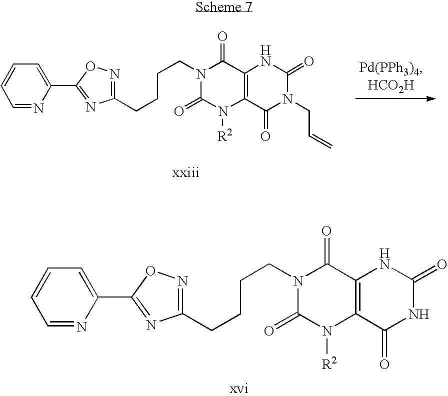

wherein R¹, R², R⁴ and R⁵ are defined above for the compounds of formula (II) and X is a good leaving group, such as —Cl, —Br, —I, —O-tosyl, —O-mesyl or —O-triflate.

A compound of formula xxiii can be treated with tetrakistriphenylphosphine palladium (IV) in the presence of formic acid to remove the allyl protecting group from the compound of formula xxiii and provide the compounds of formula xvi, which correspond to the compounds of formula (I) wherein R¹ is:

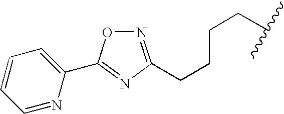

The starting materials and reagents depicted in Schemes 1-8 are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the art will recognize that the synthesis of compounds of Formula (I) may require the need for the protection of certain functional groups (i.e., derivatization for the purpose of chemical compatibility with a particular reaction condition). Suitable protecting groups for the various functional groups of the compounds of formula (I) and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

The starting materials and reagents used in preparing compounds described are either available from commercial suppliers such as Aldrich Chemical Co. (Wisconsin, USA) and Acros Organics Co. (New Jersey, USA) or were prepared using methods well-known to those skilled in the art of organic synthesis. All commercially purchased solvents and reagents were used as received. LCMS analysis was performed using an Applied Biosystems API-100 mass spectrometer equipped with a Shimadzu SCL-10A LC column: Altech platinum C18, 3 um, 33 mm×7 mm ID; gradient flow: 0 minutes, 10% CH₃CN; 5 minutes, 95% CH₃CN; 7 minutes, 95% CH₃CN; 7.5 minutes, 10% CH₃CN; 9 minutes, stop. Flash column chromatography was performed using Selecto Scientific flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

### Example 1

### Preparation of Intermediate Compounds 1B and 1C

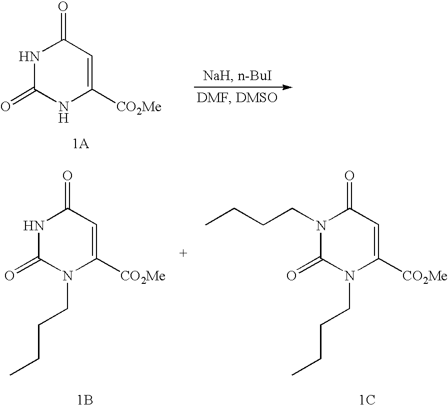

NaH (1.08 g, 24.70 mmol, 60% in mineral oil) was added to a solution of compound 1A (4.0 g, 23.52 mmol) in DMF/DMSO (30 mL/20 mL) at room temperature and the resulting mixture was stirred for 40 minutes. n-butyliodide (2.67 mL, 23.52 mmol) was then added and the reaction was stirred at room temperature for about 15 hours. The reaction mixture was then poured into a mixture of EtOAc (250 mL) and 0.5 N HCl and the organic phase was washed sequentially with water (3×) and brine, then dried (Na₂SO₄), filtered and concentrated in vacuo. The resulting residue was purified using silica gel flash column chromatography (eluting with hexane/EtOAc (v/v=1/1)) to provide monoalkylated compound 1B (1.4 g, 26%) and dialkylated product 1C (1.7 g, 26%). Electrospray MS [M+1]⁺240.1,

### Example 2

### Preparation of Intermediate Compound 2B

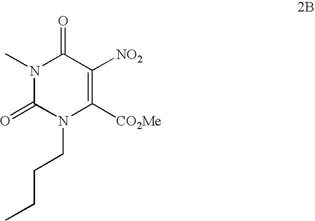

### Step A—Synthesis of Compound 2A

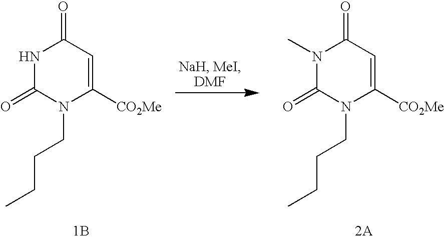

NaH (92.9 mg, 2.12 mmol, 60% in mineral oil) was added to a solution of compound 1B (0.4 g, 1.77 mmol) in DMF (3.5 mL) at room temperature and the resulting mixture was stirred for 40 minutes. Iodomethane (0.165 mL, 2.65 mmol) was then added and the reaction mixture was stirred at room temperature overnight, then poured into a mixture of EtOAc (100 mL) and 0.5 N HCl. The organic phase was washed sequentially with water (3×) and brine, then dried (Na₂SO₄), filtered and concentrated in vacuo. The resulting residue was purified using silica gel flash column chromatography (eluting with hexane/EtOAc (v/v=2/1)) to provide compound 2A (0.39 g, 92%).

### Step B—Synthesis of Compound 2B

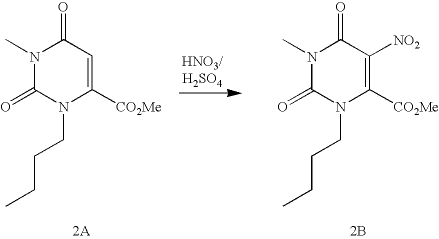

To a 0° C. solution of compound 2A (0.39 g, 1.62 mmol) in H₂SO₄ (2.4 mL, 96%), was added HNO₃ (0.6 mL, fume) dropwise. The mixture was stirred at 0° C. for 1 hour, and then warmed to room temperature and allowed to stir at this temperature for an additional 1.5 hours. The reaction mixture was then poured into EtOAc/ice. The organic phase was washed sequentially with water and brine, then dried (Na₂SO₄), filtered and concentrated in vacuo to provide compound 2B (0.39 g, 84%), which was used without further purification. Elecrospray MS [M+1]⁺285.1.

### Example 3

### Preparation of Intermediate Compound 3A

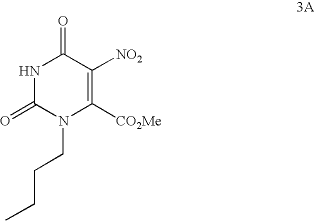

Compound 3A was prepared using the method described in Example 2, Step B and substituting compound 1B for compound 2A. Electrospray MS [M+1]⁺271.1.

### Example 4

### Preparation of Compound 1

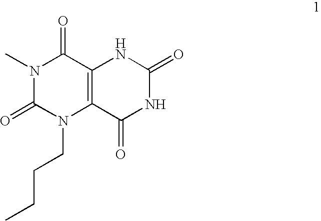

To a solution of compound 2B (0.1 g, 0.35 mmol) in HOAc (2.5 mL) was added zinc powder (225 mg, 3.5 mmol) and the resulting mixture was heated to 65° C. and allowed to stir at this temperature for 6 hours. The reaction mixture was then cooled to room temperature and filtered through a short pad of celite. The collected solid was washed with EtOH and the filtrate and washings were combined and concentrated in vacuo to provide a liquid residue to which was added powdered urea (105 mg, 1.75 mmol) and the resulting solution was heated to about 208° C. and allowed to stir at this temperature for 45 minutes. The reaction mixture was then cooled to room temperature and purified using Gilson reverse phase HPLC to provide compound 1 (14 mg, 15%). Electrospray MS [M+1]⁺267.1.

### Example 5

### Preparation of Compound 2

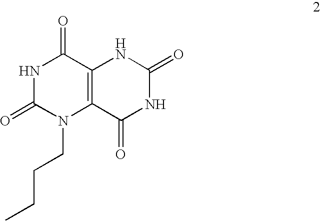

Compound 2 was prepared using the method described in Example 4 and substituting compound 3A for compound 2B. Electrospray MS [M+1]⁺253.1.

### Example 6

### Preparation of Compound 3

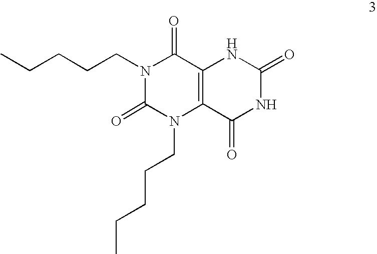

### Step A—Synthesis of Intermediate Compound 6A

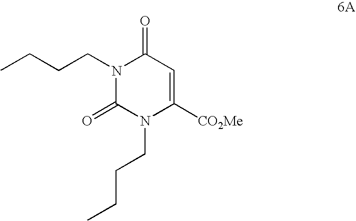

Compound 6A was prepared using the method described in Example 1 and substituting n-pentyliodide for n-butyliodide.

### Step B—Synthesis of Intermediate Compound 6B

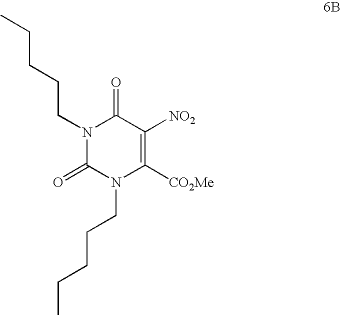

Compound 6B was prepared using the method described in Example 2, Step B, and substituting compound 6A for compound 2A.

### Step C—Preparation of Compound 3

Compound 3 was prepared by reacting compound 6A with compound 6B according to the method set forth in Example 4. Electrospray MS [M+1]⁺308.1

### Example 7

### Nicotinic Acid Receptor Assay

The nicotinic acid receptor agonist activity of the inventive compounds can be determined by following the inhibition of forskolin-stimulated cAMP accumulation in cells using the MesoScale Discovery cAMP detection kit following the manufacturer's protocol. Briefly, Chinese Hamster Ovary (CHO) cells expressing recombinant human nicotinic acid receptor (NAR) are harvested enzymatically, washed 1× in phosphate buffered saline (PBS) and resuspended in PBS containing 0.5 mM MMX at 3×10⁶ cells/mL. Ten μL of cell suspension is added to each well of a 384-well plate, each well containing 10 μL of test compound. Test compounds are diluted with PBS containing 6 μM of forskolin. Plates are incubated for 30 minutes at room temperature after the addition of cells. Lysis buffer containing cAMP-Tag is then added to each well (10 μL/well) as per the manufacturer's protocol. Plates are then incubated from 45 minutes to overnight. Prior to reading, 10 μL of read buffer is added to each well, and the plate is read in a Sector 6000 plate imager. The signal can be converted to cAMP concentration using a standard curve run on each plate. Compound EC₅₀ values can then determined from concentration gradients of test compounds.

### Uses of the Pyrimidinedione Derivatives

The Pyrimidinedione Derivatives are useful in human and veterinary medicine for treating or preventing a Condition in a patient. In accordance with the invention, the Pyrimidinedione Derivatives can be administered to a patient in need of treatment or prevention of a Condition.

### Methods For Treating or Preventing Pain

The Pyrimidinedione Derivatives are useful for treating or preventing pain in a patient.

Accordingly, in one embodiment, the present invention provides a method for treating or preventing pain in a patient, comprising administering to the patient an effective amount of one or more Pyrimidinedione Derivatives.

Illustrative examples of pain treatable or preventable using the present methods, include, but are not limited to acute pain, chronic pain, neuropathic pain, nociceptive pain, cutaneous pain, somatic pain, visceral pain, phantom limb pain, cancer pain (including breakthrough pain), pain caused by drug therapy (such as cancer chemotherapy), headache (including migraine, tension headache, cluster headache, pain caused by arithritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

### Methods for Treating or Preventing Diabetes

The Pyrimidinedione Derivatives are useful for treating or preventing diabetes in a patient. Accordingly, in one embodiment, the present invention provides a method for treating diabetes in a patient, comprising administering to the patient an effective amount of one or more Pyrimidinedione Derivatives.

Examples of diabetes treatable or preventable using the Pyrimidinedione Derivatives include, but are not limited to, type I diabetes (insulin-dependent diabetes mellitus), type II diabetes (non-insulin dependent diabetes mellitus), gestational diabetes, autoimmune diabetes, insulinopathies, idiopathic type I diabetes (Type 1b), latent autoimmumne diabetes in adults, early-onset type 2 diabetes (EOD), youth-onset atypical diabetes (YOAD), maturity onset diabetes of the young (MODY), malnutrition-related diabetes, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by □-cell toxins, and diabetes induced by drug therapy (such as diabetes induced by antipsychotic agents).

In one embodiment, the diabetes is type I diabetes.

In another embodiment, the diabetes is type II diabetes.

### Methods for Treating or Preventing a Diabetic Complication

The Pyrimidinedione Derivatives are useful for treating or preventing a diabetic complication in a patient. Accordingly, in one embodiment, the present invention provides a method for treating a diabetic complication in a patient, comprising administering to the patient an effective amount of one or more Pyrimidinedione Derivatives.

Examples of diabetic complications treatable or preventable using the present methods include, but are not limted to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, hypertension, syndrome of insulin resistance, coronary artery disease, a fungal infection, a bacterial infection, and cardiomyopathy.

### Methods for Treating or Preventing Impaired Glucose Tolerance

The Pyrimidinedione Derivatives are useful for treating or preventing impaired glucose tolerance in a patient.

Accordingly, in one embodiment, the present invention provides a method for treating impaired glucose tolerance in a patient, comprising administering to the patient an effective amount of one or more Pyrimidinedione Derivatives.

### Methods for Treating or Preventing Impaired Fasting Glucose

The Pyrimidinedione Derivatives are useful for treating or preventing impaired fasting glucose in a patient.

Accordingly, in one embodiment, the present invention provides a method for treating impaired fasting glucose in a patient, comprising administering to the patient an effective amount of one or more Pyrimidinedione Derivatives.

### Methods for Treating or Preventing Obesity

The Pyrimidinedione Derivatives are useful for treating or preventing obesity or an obesity-related disorder in a patient.

Accordingly, in one embodiment, the present invention provides a method for treating obesity or an obesity-related disorder in a patient, comprising administering to the patient an effective amount of one or more Pyrimidinedione Derivatives.

### Methods for Treating or Preventing a Hematological Disorder

The Pyrimidinedione Derivatives are useful for treating or preventing a hematological disorder in a patient.

Accordingly, in one embodiment, the present invention provides a method for treating a hematological disorder in a patient, comprising administering to the patient an effective amount of one or more Pyrimidinedione Derivatives.

Examples of hematological disorders treatable or preventable using the present methods include, but are not limted to, an anemia caused by hemolysis, an anemia caused by deficient erythropoiesis, a coagulation disorder, an eosinophilic disorder, hemostasis, a histiocytic syndrome, neutropenia, lymphocytopenia, thrombocytopenia, a thrombic disorder, a platelet disorder or a clotting disorder.

### Methods for Treating or Preventing a Neurological Disorder

The Pyrimidinedione Derivatives are useful for treating or preventing a neurological disorder in a patient.

Accordingly, in one embodiment, the present invention provides a method for treating a neurological disorder in a patient, comprising administering to the patient an effective amount of one or more Pyrimidinedione Derivatives.

Examples of neurological disorders treatable or preventable using the present methods include, but are not limted to, meningitis, a movement disorder (such as Parkinson's disease or Huntington's disease), delirium, dementia, a demyelinating disorder (such as multiple sclerosis or amyotrophic lateral sclerosis), aphasia, a peripheral nervous system disorder, a seizure disorder, a sleep disorder, a spinal cord disorder or stroke.

### Methods for Treating or Preventing a Cardiovascular Disease

The Pyrimidinedione Derivatives are useful for treating or preventing a cardiovascular disease in a patient.

Accordingly, in one embodiment, the present invention provides a method for treating a cardiovascular disease in a patient, comprising administering to the patient an effective amount of one or more Pyrimidinedione Derivatives.

Illustrative examples of cardiovascular diseases treatable or preventable using the present methods, include, but are not limited to atherosclerosis, congestive heart failure, cardiac arrhythmia, myocardial infarction, atrial fibrillation, atrial flutter, circulatory shock, left ventricular hypertrophy, ventricular tachycardia, supraventricular tachycardia, coronary artery disease, angina, infective endocarditis, non-infective endocarditis, cardiomyopathy, peripheral artery disease, Reynaud's phenomenon, deep venous thrombosis, aortic stenosis, mitral stenosis, pulmonic stenosis and tricuspid stenosis.

In one embodiment, the cardiovascular disease is atherosclerosis.

In another embodiment, the cardiovascular disease is congestive heart failure.

In another embodiment, the cardiovascular disease is coronary artery disease.

### Methods for Treating or Preventing a Respiratory Disorder

The Pyrimidinedione Derivatives are useful for treating or preventing a respiratory disorder in a patient.

Accordingly, in one embodiment, the present invention provides a method for treating a respiratory disorder in a patient, comprising administering to the patient an effective amount of one or more Pyrimidinedione Derivatives.

Examples of respiratory disorders treatable or preventable using the present methods include, but are not limted to, asthma, bronchiectasis, chronic obstructive pulmonary disease, an interstitial lung disease, a mediastal disorder, a pleural disorder, pneumonia or sarcoidosis.

### Methods for Treating or Preventing a Gastroenterological Disorder

The Pyrimidinedione Derivatives are useful for treating or preventing a gastroenterological disorder in a patient.

Accordingly, in one embodiment, the present invention provides a method for treating a gastroenterological disorder in a patient, comprising administering to the patient an effective amount of one or more Pyrimidinedione Derivatives.

Examples of gastroenterological disorders treatable or preventable using the present methods include, but are not limted to, an anorectal disorder, diarrhea, irritable bowel syndrome, dyspepsis, gastroesophageal reflux disease, diverticulitis, gastritis, peptic ulcer disease, gastroenteritis, inflammatory bowel disease, a malabsorption syndrome or pancreatitis.

### Methods for Treating or Preventing Inflammation

The Pyrimidinedione Derivatives are useful for treating or preventing inflammation in a patient.

Accordingly, in one embodiment, the present invention provides a method for treating inflammation in a patient, comprising administering to the patient an effective amount of one or more Pyrimidinedione Derivatives.

### Methods for Treating or Preventing Non-Alcoholic Fatty Liver Disease

The Pyrimidinedione Derivatives are useful for treating or preventing non-alcoholic fatty liver disease in a patient.

Accordingly, in one embodiment, the present invention provides a method for treating non-alcoholic fatty liver disease in a patient, comprising administering to the patient an effective amount of one or more Pyrimidinedione Derivatives.

### Methods for Treating or Preventing Dyslipidemia

The Pyrimidinedione Derivatives are useful for treating or preventing dyslipidemia in a patient.

Accordingly, in one embodiment, the present invention provides a method for treating dyslipidemia in a patient, comprising administering to the patient an effective amount of one or more Pyrimidinedione Derivatives.

### Methods for Treating or Preventing a Metabolic Disorder

The Pyrimidinedione Derivatives can also be useful for treating a metabolic disorder. Examples of metabolic disorders treatable include, but are not limited to, metabolic syndrome (also known as “Syndrome X”), impaired glucose tolerance, impaired fasting glucose, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

Accordingly, in one embodiment, the invention provides methods for treating a metabolic disorder in a patient, wherein the method comprises administering to the patient an effective amount of one or more Pyrimidinedione Derivatives, or a pharmaceutically acceptable salt, solvate, ester, prodrug or stereoisomer thereof.

In one embodiment, the metabolic disorder is hypercholesterolemia.

In another embodiment, the metabolic disorder is hyperlipidemia.

In another embodiment, the metabolic disorder is hypertriglyceridemia.

In still another embodiment, the metabolic disorder is metabolic syndrome.

In a further embodiment, the metabolic disorder is low HDL levels.

### Methods for Treating or Preventing Cancer

The Pyrimidinedione Derivatives are useful for treating or preventing cancer in a patient.

Accordingly, in one embodiment, the present invention provides a method for treating cancer in a patient, comprising administering to the patient an effective amount of one or more Pyrimidinedione Derivatives.

Non-limiting examples of cancers treatable or preventable using the present methods include the following cancers and metastases thereof: bladder cancer, breast cancer, colorectal cancer, kidney cancer, liver cancer, non-small cell lung cancer, small cell lung cancer, non-small cell lung cancer, head and neck cancer, esophageal cancer, gall bladder cancer, ovarian cancer, pancreatic cancer, stomach cancer, cervical cancer, thyroid cancer, prostate cancer, skin cancer; hematopoietic tumors of lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including brain tumors (such as an astrocytoma, a neuroblastoma, a glioma or a schwannoma); and other tumors, including melanoma, seminoma, teratocarcinoma, osteosarcoma, xenoderoma pigmentosum, keratoctanthoma, thyroid follicular cancer and Kaposi's sarcoma. The Pyrimidinedione Derivatives are useful for treating primary tumors, metastatic tumors and tumors of unknown origin.

In one embodiment, the cancer treated is lung cancer.

In another embodiment, the cancer treated is breast cancer.

In another embodiment, the cancer treated is colorectal cancer.

In still another embodiment, the cancer treated is prostate cancer.

In another embodiment, the cancer treated is a leukemia.

In yet another embodiment, the cancer treated is a lymphoma.

In a further embodiment, the cancer treated is a metastatic tumor.

In one embodiment, the Pyrimidinedione Derivatives can be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

In another embodiment, the Pyrimidinedione Derivatives can be useful in inhibiting tumor angiogenesis and metastasis.

### Combination Therapy

In one embodiment, the present invention provides methods for treating a Condition in a patient, the method comprising administering to the patient one or more Pyrimidinedione Derivatives, or a pharmaceutically acceptable salt, solvate, ester, prodrug or stereoisomer thereof and at least one additional therapeutic agent that is not a Pyrimidinedione Derivative, wherein the amounts administered are together effective to treat or prevent a Condition.

Non-limiting examples of additional therapeutic agents useful in the present methods for treating or preventing a Condition include an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, an ACAT inhibitors, a cholesteryl ester transfer protein (CETP) inhibitor, a low-density lipoprotein (LDL) activator, or any combination of two or more of these additional therapeutic agents.

Further non-limiting examples of additional therapeutic agents useful in the present methods for treating or preventing a condition include hydroxy-substituted azetidinone compounds, substituted β-lactam compounds, α-amylase inhibitors, α-glucoside hydrolase inhibitors, fatty acid oxidation inhibitors, A2 antagonists, c-jun amino-terminal kinase inhibitors, glycogen phosphorylase inhibitors, VPAC2 receptor agonists, glucokinase activators, nicotinic acid receptor antagonists, bile acid sequestrants, inorganic cholesterol sequestrants, AcylCoA:Cholesterol O-acyltransferase inhibitors, cholesteryl ester transfer protein inhibitors, fish oils containing Omega 3 fatty acids, natural water soluble fibers, plant stanols and/or fatty acid esters of plant stanols, anti-oxidants, FXR receptor modulators, LXR receptor agonists, lipoprotein synthesis inhibitors, renin angiotensin inhibitors, microsomal triglyceride transport protein inhibitors, bile acid reabsorption inhibitors, triglyceride synthesis inhibitors, squalene epoxidase inhibitors, low density lipoprotein receptor inducers or activators, platelet aggregation inhibitors, 5-LO or FLAP inhibitors, PPAR δ partial agonists, 5HT transporter inhibitors, NE transporter inhibitors, ghrelin antagonists, H₃ antagonists/inverse agonists, MCH1R antagonists, MCH2R agonists/antagonists, leptin agonists/modulators, leptin derivatives, opioid antagonists, orexin receptor antagonists, BRS3 agonists, CCK-A agonists, CNTF, CNTF derivatives, CNTF agonists/modulators, 5HT2c agonists, Mc4r agonists, monoamine reuptake inhibitors, serotonin reuptake inhibitors, phentermine, topiramate, phytopharm compound 57, ghrelin antibodies, Mc3r agonists, ACC inhibitors, β3 agonists, DGAT1 inhibitors, DGAT2 inhibitors, FAS inhibitors, PDE inhibitors, thyroid hormone β agonists, UCP-1 activators, UCP-2 activators, UCP-3 activators, acyl-estrogens, glucocorticoid agonists/antagonists, lipase inhibitors, fatty acid transporter inhibitors, dicarboxylate transporter inhibitors, glucose transporter inhibitors, phosphate transporter inhibitorsanti-hypertensive agents, anti-dyslipidemic agents, DP receptor antagonists, apolipoprotein-B secretion/microsomal triglyceride transfer protein (apo-B/MTP) inhibitors, sympathomimetic agonists, dopamine agonists, melanocyte-stimulating hormone receptor analogs, leptons, galanin receptor antagonists, bombesin agonists, thyromimetic agents, dehydroepiandrosterone, analogs of dehydroepiandrosterone, urocortin binding protein antagonists, human agouti-related proteins (AGRP), neuromedin U receptor agonists, noradrenergic anorectic agents, hormone sensitive lipase antagonists, MSH-receptor analogs, α-glucosidase inhibitors, apo A1 milano reverse cholesterol transport inhibitors, fatty acid binding protein inhibitors (FABP), fatty acid transporter protein inhibitors (FATP), an antihypertensive agent.

Examples of antidiabetic agents useful in the present methods for treating or preventing a Condition include, but are not limited to: a sulfonylurea, an insulin sensitizer, a glucosidase inhibitor, an insulin secretagogue, a hepatic glucose output lowering agent, an anti-obesity agent, an antihypertensive agent, a meglitinide, an agent that slows or blocks the breakdown of starches and sugars in vivo, a histamine H₃ receptor antagonist, an antihypertensive agent, a sodium glucose uptake transporter 2 (SGLT-2) inhibitor, a peptide that increases insulin production, and insulin or any insulin-containing composition.

In one embodiment, the antidiabetic agent is an insulin sensitizer.

Non-limiting examples of insulin sensitizers include PPAR activators, such as the glitazone and thiazoldinedione class of agents, which include rosiglitazone, rosiglitazone maleate (AVANDIA™ from GlaxoSmithKline), pioglitazone, pioglitazone hydrochloride (ACTOS™, from Takeda) ciglitazone and MCC-555 (Mitstubishi Chemical Co.), troglitazone and englitazone; biguanides, such as phenformin, metformin, metformin hydrochloride (such as GLUCOPHAGE® from Bristol-Myers Squibb), metformin hydrochloride with glyburide (such as GLUCOVANCE™ from Bristol-Myers Squibb) and buformin; DPP-IV inhibitors, such as sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck); PTP-1B inhibitors, such as A-401,674, KR 61639, OC-060062, OC-83839, OC-297962, MC52445, and MC52453; and α-glucokinase activators, such as acarbose, adipose, camiglibose, emiglitate, miglitol, voglibose, pradimicin-Q, salbostatin, CDK-711, MDL-25,637, MDL-73,945, and MOR 14.

In one embodiment, the antidiabetic agent is a DPP-IV inhibitor.

In another embodiment, the antidiabetic agent is a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

In one embodiment, the antidiabetic agent is a SGLT-2 inhibitor.

Non-limiting examples of SGLT-2 inhibitors useful in the present methods include dapagliflozin and sergliflozin, AVE2268 (Sanofi-Aventis) and T-1095 (Tanabe Seiyaku).

In another embodiment, the antidiabetic agent is a hepatic glucose output lowering agent.

Non-limiting examples of hepatic glucose output lowering agents include Glucophage and Glucophage XR.

In one embodiment, the antidiabetic agent is an insulin secretagogue.

Non-limiting examples of insulin secretagogues include GLP-1, GLP-1 mimetics, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

In another embodiment, the antidiabetic agent is insulin or an insulin-containing preparation.

The term “insulin” as used herein, includes all formualtions of insulin, including long acting and short acting forms of insulin.

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In one embodiment, the antidiabetic agent is anti-obesity agent, including, but not limited to those set forth below herein.

In another embodiment, the antidiabetic agent is an antihypertensive agent.

Non-limiting examples of antihypertensive agents useful in the present methods for treating diabetes include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

In another embodiment, the antidiabetic agent is a meglitinide.

Non-limiting examples of meglitinides useful in the present methods for treating diabetes include repaglinide and nateglinide.

In still another embodiment, the antidiabetic agent is an agent that slows or blocks the breakdown of starches and sugars in vivo.

Non-limiting examples of antidiabetic agents that slow or block the breakdown of starches and sugars in vivo and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

Non-limiting examples of anti-obesity agents useful in the present methods for treating a Condition include an appetite suppressant; a 5-HT2C agonist, such as lorcaserin; an AMP kinase activator; a histamine H₃ receptor antagonist or inverse agonist; a metabolic rate enhancer; or a nutrient absorption inhibitor.

Non-limiting examples of appetite suppressant agents useful in the present methods for treating or preventing a Condition include cannabinoid receptor 1 (CB_{I}) antagonists or inverse agonists (e.g., rimonabant); Neuropeptide Y (NPY1, NPY2, NPY4 and NPY5) antagonists; metabotropic glutamate subtype 5 receptor (mGluR5) antagonists (e.g., 2-methyl-6-(phenylethynyl)-pyridine and 3[(2-methyl-1,4-thiazol-4-yl)ethynyl]pyridine); melanin-concentrating hormone receptor (MCH1R and MCH2R) antagonists; melanocortin receptor agonists (e.g., Melanotan-II and Mc4r agonists); serotonin uptake inhibitors (e.g., dexfenfluramine and fluoxetine); serotonin (5HT) transport inhibitors (e.g., paroxetine, fluoxetine, fenfluramine, fluvoxamine, sertaline and imipramine); norepinephrine (NE) transporter inhibitors (e.g., desipramine, talsupram and nomifensine); ghrelin antagonists; leptin, adiponectin, or derivatives thereof; opioid antagonists (e.g., nalmefene, 3-methoxynaltrexone, naloxone and nalterxone); orexin antagonists; bombesin receptor subtype 3 (BRS3) agonists; Cholecystokinin-A (CCK-A) agonists; ciliary neurotrophic factor (CNTF) or derivatives thereof (e.g., butabindide and axokine); monoamine reuptake inhibitors (e.g., sibutramine); glucagon-like peptide 1 (GLP-1) agonists; topiramate; and phytopharm compound 57.

Non-limiting examples of metabolic rate enhancers useful in the present methods for treating or preventing a Condition include acetyl-CoA carboxylase-2 (ACC2) inhibitors; beta adrenergic receptor 3 (β3) agonists; diacylglycerol acyltransferase inhibitors (DGAT1 and DGAT2); fatty acid synthase (FAS) inhibitors (e.g., Cerulenin); phosphodiesterase (PDE) inhibitors (e.g., theophylline, pentoxifylline, zaprinast, sildenafil, aminone, milrinone, cilostamide, rolipram and cilomilast); thyroid hormone β agonists; uncoupling protein activators (UCP-1, 2 or 3) (e.g., phytanic acid, 4-[(E)-2-(5,6,7,8-tetramethyl-2-naphthalenyl)-1-propenyl]benzoic acid and retinoic acid); acyl-estrogens (e.g., oleoyl-estrone); glucocorticoid antagonists; 11-beta hydroxy steroid dehydrogenase type 1 (11β HSD-1) inhibitors; melanocortin-3 receptor (Mc3r) agonists; and stearoyl-CoA desaturase-1 (SCD-1) compounds.

Non-limiting examples of nutrient absorption inhibitors useful in the present methods for treating or preventing a Condition include lipase inhibitors (e.g., orlistat, lipstatin, tetrahydrolipstatin, teasaponin and diethylumbelliferyl phosphate); fatty acid transporter inhibitors; dicarboxylate transporter inhibitors; glucose transporter inhibitors; and phosphate transporter inhibitors.

Non-limiting examples of H₃ antagonists/inverse agonists useful in combination with the Pyrimidinedione Derivatives include thioperamide, 3-(1H-imidazol-4-yl)propyl N-(4-pentenyl)carbamate, clobenpropit, iodophenpropit, imoproxifan, and GT2394 (Gliatech), those described in WO 02/15905 (herein incorporated by reference); O-[3-(1H-imidazol-4-yl)propanol]carbamates described in Kiec-Kononowicz, K. et al., Pharmazie, 55:349-55 (2000) (herein incorporated by reference), piperidine-containing histamine H₃-receptor antagonists described in Lazewska, D. et al., Pharmazie, 56:927-32 (2001) (herein incorporated by reference), benzophenone derivatives and related compounds described in Sasse, A. et al., Arch. Pharm. (Weinheim) 334:45-52 (2001)(herein incorporated by reference), substituted N-phenylcarbamates described in Reidemeister, S. et al., Pharmazie, 55:83-6 (2000) (herein incorporated by reference), and proxifan derivatives described in Sasse, A. et al., J. Med. Chem. 43:3335-43 (2000) (each of the preceding references is herein incorporated by reference), and the following compound:

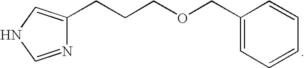

Non-limiting examples of cholesterol biosynthesis inhibitors useful in the present methods for treating or preventing a Condition include HMG-CoA reductase inhibitors, squalene synthase inhibitors, squalene epoxidase inhibitors, and mixtures thereof.

Non-limiting examples of cholesterol absorption inhibitors useful in the present methods for treating or preventing a Condition include ezetimibe. In one embodiment, the cholesterol absorption inhibitor is ezetimibe.

Non-limiting examples of squalene synthesis inhibitors useful in the present methods for treating or preventing a Condition include, but are not limited to, squalene synthetase inhibitors, such as squalestatin 1; and squalene epoxidase inhibitors, such as NB-598 ((E)-N-ethyl-N-(6,6-dimethyl-2-hepten-4-ynyl)-3-[(3,3′-bithiophen-5-yl)methoxy]benzene-methanamine hydrochloride).

Non-limiting examples of bile acid sequestrants useful in the present methods for treating or preventing a Condition include, but are not limited to, cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Suitable inorganic cholesterol sequestrants include bismuth salicylate plus montmorillonite clay, aluminum hydroxide and calcium carbonate antacids.

Probucol derivatives useful in the present methods for treating or preventing a Condition include, but are not limited to, AGI-1067 and others disclosed in U.S. Pat. Nos. 6,121,319 and 6,147,250.

IBAT inhibitors useful in the present methods for treating or preventing a Condition include, but are not limited to, benzothiepines such as therapeutic compounds comprising a 2,3,4,5-tetrahydro-1-benzothiepine 1,1-dioxide structure such as are disclosed in International Publication No. WO 00/38727.

Nicotinic acid derivatives useful in the present methods for treating or preventing a Condition include, but are not limited to, those having a pyridine-3-carboxylate structure or a pyrazine-2-carboxylate structure, including acid forms, salts, esters, zwitterions and tautomers, where available. Other examples of nicotinic acid derivatives useful in the present methods include nicotinic acid, niceritrol, nicofuranose and acipimox (5-methylpyrazine-2-carboxylic acid 4-oxide). An example of a suitable nicotinic acid product is NIASPAN® (niacin extended-release tablets) which are available from Kos Pharmaceuticals, Inc. (Cranbury, N.J.). Further nicotinic acid derivatives useful in the present methods for treating or preventing a Condition include, but are not limited to, the compounds disclosed in U.S. Patent Publication Nos. 2006/0264489 and 2007/0066630, and U.S. patent application Ser. No. 11/771,538, each of which is incorporated herein by reference.

LDL-receptor activators useful in the present methods for treating or preventing a Condition include, but are not limited to, include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Natural water-soluble fibers useful in the present methods for treating or preventing a Condition include, but are not limited to, psyllium, guar, oat and pectin.

Fatty acid esters of plant stanols useful in the present methods for treating or preventing a Condition include, but are not limited to, the sitostanol ester used in BENECOL® margarine.

Non-limiting examples of hydroxy-substituted azetidinone compounds and substituted β-lactam compounds useful in the present methods for treating or preventing a Condition include those disclosed in U.S. Pat. Nos. 5,767,115, 5,624,920, 5,668,990, 5,656,624 and 5,688,787, 5,756,470, U.S. Patent Application Nos. 2002/0137690 and 2002/0137689 and PCT Patent Application No. WO 2002/066464, each of which is incorporated herein by reference in their entirety. A preferred azetidinone compound is ezetimibe (for example, ZETIA® which is available from Schering-Plough Corporation).

Non-limiting examples of HMG-CoA reductase inhibitors useful in the present methods for treating or preventing a Condition include lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin, fluvastatin, cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), pitavastatin (such as NK-104 of Negma Kowa of Japan).

A non-limiting example of a HMG-CoA synthetase inhibitor useful in combination with the Pyrimidinedione Derivatives is, for example, L-659,699 ((E,E)-11-[3′R-(hydroxy-methyl)-4′-oxo-2′R-oxetanyl]-3,5,7R-trimethyl-2,4-undecadienoic acid).

Non-limiting examples of AcylCoA:Cholesterol O-acyltransferase (“ACAT”) inhibitors useful in the present methods for treating or preventing a Condition include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60(1); 55-93, which is incorporated by reference herein.

Non-limiting examples of cholesteryl ester transfer protein (“CETP”) inhibitors useful in the present methods for treating or preventing a Condition include those disclosed in PCT Patent Application No. WO 00/38721, U.S. Pat. Nos. 6,147,090, 6,958,346, 6,924,313 6,906,082, 6,861,561, 6,803,388, 6,794,396, 6,787,570, 6,753,346, 6,723,752, 6,723,753, 6,710,089, 6,699,898, 6,696,472, 6,696,435, 6,683,113, 5,519,001, 5,512,548, 6,410,022, 6,426,365, 6,448,295, 6,387,929, 6,683,099, 6,677,382, 6,677,380, 6,677,379, 6,677,375, 6,677,353, 6,677,341, 6,605,624, 6,586,433, 6,451,830, 6,451,823, 6,462,092, 6,458,849, 6,458,803, 6,455,519, 6,583,183, 6,562,976, 6,555,113, 6,544,974, 6,521,607, 6,489,366, 6,482,862, 6,479,552, 6,476,075, 6,476,057, and 6,897,317, each of which are incorporated herein by reference; compounds described in Yan Xia et al., “Substituted 1,3,5-Triazines As Cholesteral Ester Transfer Protein Inhibitors”, Bioorganic & Medicinal Chemistry Letters, vol. 6, No. 7, 1996, pp. 919-922, herein incorporated by reference; natural products described in S. Coval et al., “Wiedendiol-A and -B, Cholesteryl Ester Transfer Protein Inhibitors From The Marine Sponge Xestosponga Wiedenmayeri”, Bioorganic & Medicinal Chemistry Letter, vol. 5, No. 6, pp. 605-610, 1995, herein incorporated by reference; the compounds described in Barrett et al. J. Am. Chem. Soc., 188, 7863-63 (1996), herein incorporated by reference; the compounds described in Kuo et al. J. Am. Chem. Soc., 117, 10629-34 (1995), herein incorporated by reference; the compounds described in Pietzonka et al. Bioorg. Med. Chem. Lett., 6, 1951-54 (1996), herein incorporated by reference; the compounds described in Lee et al. J. Antibiotics, 49, 693-96 (1996), herein incorporated by reference; the compounds described by Busch et al. Lipids, 25, 216-220, (1990), herein incorporated by reference; the compounds described in Morton and Zilversmit J. Lipid Res., 35, 836-47 (1982), herein incorporated by reference; the compounds described in Connolly et al. Biochem. Biophys. Res. Comm., 223, 42-47 (1996), herein incorporated by reference; the compounds described in Bisgaier et al. Lipids, 29, 811-8 (1994), herein incorporated by reference; the compounds described in EP 818448, herein incorporated by reference; the compounds described in JP 10287662, herein incorporated by reference; the compounds described in PCT applications WO 98/35937, WO 9914174, WO 9839299, and WO 9914215, each of which is herein incorporated by reference; the compounds of EP applications EP 796846, EP 801060, 818448, and 818197, each of which is herein incorporated by reference; probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE-402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13:1005-12, herein incorporated by reference; 4-carboxyamino-2-substituted-1,2,3,4-tetrahydroquinolines, e.g., those described in WO 00/017164, WO 00/017166, WO 00/140190, WO 00/213797, and WO 2005/033082 (each of which is herein incorporated by reference). These 4-carboxyamino-2-substituted-1,2,3,4-tetrahydroquinolines can be combined with HMG-CoA reductase inhibitors such as atorvastatin (WO 00/213797, WO 2004/056358, WO 2004/056359, and WO2005/011634).

A non-limiting example of a fish oil containing Omega 3 fatty acids useful in combination with the Pyrimidinedione Derivatives is 3-PUFA.

Non-limiting examples of natural water soluble fibers useful in the present methods for treating or preventing a Condition include psyllium, guar, oat and pectin.

A non-limiting example of a plant stanol and/or fatty acid ester of plant stanols useful in combination with the Pyrimidinedione Derivatives is the sitostanol ester used in BENECOL® margarine.

A non-limiting example of an anti-oxidant useful in combination with the Pyrimidinedione Derivatives includes probucol.

Non-limiting examples of NE (norepinephrine) transport inhibitors useful in combination with the Pyrimidinedione Derivatives include GW 320659, despiramine, talsupram, and nomifensine.

Non-limiting examples of CB₁ antagonists/inverse agonists useful in combination with the Pyrimidinedione Derivatives include rimonabant, SR-147778 (Sanofi Aventis), and the compounds described in U.S. Pat. No. 5,532,237, U.S. Pat. No. 4,973,587, U.S. Pat. No. 5,013,837, U.S. Pat. No. 5,081,122, U.S. Pat. No. 5,112,820, U.S. Pat. No. 5,292,736, U.S. Pat. No. 5,624,941, U.S. Pat. No. 6,028,084, WO 96/33159, WO 98/33765, WO 98/43636, WO 98/43635, WO 01/09120, WO 98/31227, WO 98/41519, WO 98/37061, WO 00/10967, WO 00/10968, WO 97/29079, WO 99/02499, WO 01/58869, WO 02/076949, and EP-658546 (each of the preceding references is herein incorporated by reference).

Non-limiting examples of ghrelin antagonists useful in combination with the Pyrimidinedione Derivatives include those described in WO 01/87335 and WO 02/08250 (each of the preceding references is herein incorporated by reference). Ghrelin antagonists are also known as GHS (growth hormone secretagogue receptor) antagonists. The pharmaceutical combinations and methods of the present invention therefore comprehend the use GHS antagonists in place of ghrelin antagonists (in combination with the nicotinic acid receptor agonists of the present invention).

Non-limiting examples of MCH1R (melanin-concentrating hormone 1 receptor) antagonists and MCH2R (melanin-concentrating hormone 2 receptor) agonists/antagonists useful in combination with the Pyrimidinedione Derivatives include those described in WO 01/82925, WO 01/87834, WO 02/06245, WO 02/04433, WO 02/51809, and JP 13226269 (each of the preceding references is herein incorporated by reference), and T-226296 (Takeda).

Non-limiting examples of NPY1 antagonists useful in combination with the Pyrimidinedione Derivatives include those described in U.S. Pat. No. 6,001,836, WO 96/14307, WO 01/23387, WO 99/51600, WO 01/85690, WO 01/85098, WO 01/85173, and WO 01/89528 (each of the preceding references is herein incorporated by reference); and BIBP3226, J-115814, BIBO 3304, LY-357897, CP-671906, and GI-264879A.

Non-limiting examples of NPY2 agonists useful in combination with the Pyrimidinedione Derivatives include PYY3-36 as described in Batterham, et al., Nature. 418:650-654 (2003), NPY3-36, and other Y2 agonists such as N acetyl [Leu(28,31)] NPY 24-36 (White-Smith and Potter, Neuropeptides 33:526-33 (1999)), TASP-V (Malis et al., Br. J. Pharmacol. 126:989-96 (1999)), cyclo-(28/32)-Ac-[Lys28-Glu32]-(25-36)-pNPY (Cabrele and Beck-Sickinger J-Pept-Sci. 6:97-122 (2000)) (each of the preceding references is herein incorporated by reference).

Non-limiting examples of NPY4 agonists useful in combination with the Pyrimidinedione Derivatives include pancreatic peptide (PP) as described in Batterham et al., J. Clin. Endocrinol. Metab. 88:3989-3992 (2003), and other Y4 agonists such as 1229U91 (Raposinho et al., Neuroendocrinology. 71:2-7 (2000) (both references are herein incorporated by reference).

Non-limiting examples of NPY5 antagonists useful in combination with the Pyrimidinedione Derivatives include those described in U.S. Pat. No. 6,140,354, U.S. Pat. No. 6,191,160, U.S. Pat. No. 6,258,837, U.S. Pat. No. 6,313,298, U.S. Pat. No. 6,337,332, U.S. Pat. No. 6,329,395, U.S. Pat. No. 6,340,683, U.S. Pat. No. 6,326,375, U.S. Pat. No. 6,335,345, EP-01010691, EP-01044970, WO 97/19682, WO 97/20820, WO 97/20821, WO 97/20822, WO 97/20823, WO 98/27063, WO 00/64880, WO 00/68197, WO 00/69849, WO 01/09120, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, WO 02/49648, WO 01/14376, WO 04/110375, WO 05/000217 and Norman et al., J. Med. Chem. 43:4288-4312 (2000) (each of the preceding references is herein incorporated by reference); and 152,804, GW-569180A, GW-594884A, GW-587081×, GW-548118×; FR226928, FR 240662, FR252384; 1229U91, GI-264879A, CGP71683A, LY-377897, PD-160170, SR-120562A, SR-120819A and JCF-104.

Non-limiting examples of mGluR5 (Metabotropic glutamate subtype 5 receptor) antagonists useful in combination with the Pyrimidinedione Derivatives include 2-methyl-6-(phenylethynyl)-pyridine (MPEP) and (3-[(2-methyl-1,3-thiazol-4-yl)ethynyl]pyridine) (MTEP) and those compounds described in Anderson J. et al., J, Eur J. Pharmacol. Jul. 18, 2003; 473(1):35-40; Cosford N. et al., Bioorg Med Chem. Lett. Feb. 10, 2003; 13(3):351-4; and Anderson J. et al., J Pharmacol Exp Ther. December 2002:303(3):1044-51 (each of the preceding references is herein incorporated by reference).

Non-limiting examples of leptins, leptin derivatives, and leptin agonists/modulators useful in combination with the Pyrimidinedione Derivatives include recombinant human leptin (PEG-OB, Hoffman La Roche) and recombinant methionyl human leptin (Amgen). Leptin derivatives (e.g., truncated forms of leptin) useful in the present invention include those described in U.S. Pat. No. 5,552,524, U.S. Pat. No. 5,552,523, U.S. Pat. No. 5,552,522, U.S. Pat. No. 5,521,283, WO 96/23513, WO 96/23514, WO 96/23515, WO 96/23516, WO 96/23517, WO 96/23518, WO 96/23519, and WO 96/23520 (each of the preceding references is herein incorporated by reference).

Non-limiting examples of opioid antagonists useful in combination with the Pyrimidinedione Derivatives include nalmefene (Revex™), 3-methoxynaltrexone, naloxone, and naltrexone, as well as opioid antagonists described in WO 00/21509 (herein incorporated by reference).

Non-limiting examples of orexin receptor antagonists useful in combination with the Pyrimidinedione Derivatives include SB-334867-A, as well as those described in WO 01/96302, WO 01/68609, WO 02/51232, and WO 02/51838 (each of the preceding references is herein incorporated by reference).

Non-limiting examples of CNTF (specific ciliary neurotrophic factors) useful in combination with the Pyrimidinedione Derivatives include GI-181771 (Glaxo-SmithKline); SR146131 (Sanofi Aventis); butabindide; PD170,292, PD 149164 (Pfizer).

Non-limiting examples of CNTF derivatives and CNTF agonists/modulators useful in combination with the Pyrimidinedione Derivatives include axokine (Regeneron) and those described in WO 94/09134, WO 98/22128, and WO 99/43813 (each of which is herein incorporated by reference).

Non-limiting examples of 5HT2c agonists useful in combination with the Pyrimidinedione Derivatives include BVT933, DPCA37215, WAY161503, and R-1065, as well as those described in U.S. Pat. No. 3,914,250, WO 02/36596, WO 02/48124, WO 02/10169, WO 01/66548, WO 02/44152, WO 02/51844, WO 02/40456, and WO 02/40457 (each of which is herein incorporated by reference).

Non-limiting examples of Mc4r agonists useful in combination with the Pyrimidinedione Derivatives include CHIR86036 (Chiron); ME-10142, and ME-10145 (Melacure), as well as those described in WO 01/991752, WO 01/74844, WO 02/12166, WO 02/11715, and WO 02/12178 (each of which is herein incorporated by reference).

Non-limiting examples of monoamine reuptake inhibitors useful in combination with the Pyrimidinedione Derivatives include sibutramine (Meridia™/Reductil™), as well as those described in WO 01/27068, WO 01/62341, U.S. Pat. No. 4,746,680, U.S. Pat. No. 4,806,570, U.S. Pat. No. 5,436,272, and US 2002/0006964 (each of which is herein incorporated by reference).

Non-limiting examples of serotonin reuptake inhibitors useful in combination with the Pyrimidinedione Derivatives include dexfenfluramine, fluoxetine, and those described in U.S. Pat. No. 6,365,633, WO 01/27060, and WO 01/162341 (each of which is herein incorporated by reference).

Non-limiting examples of α-amylase inhibitors useful in combination with the Pyrimidinedione Derivatives include tendamistat, trestatin, and Al-3688.

Non-limiting examples of α-glucokinase activators useful in combination with the Pyrimidinedione Derivatives include acarbose, adipose, camiglibose, emiglitate, miglitol, voglibose, pradimicin-Q, salbostatin, CDK-711, MDL-25,637, MDL-73,945, and MOR 14.

Non-limiting examples of fatty acid oxidation inhibitors useful in combination with the Pyrimidinedione Derivatives include clomoxir and etomoxir.

Non-limiting examples of A2 antagonists useful in combination with the Pyrimidinedione Derivatives include midaglizole, isaglidole, deriglidole, idazoxan, earoxan, and fluparoxan.

Non-limiting examples of glycogen phosphorylase inhibitors useful in combination with the Pyrimidinedione Derivatives include CP-368,296, CP-316,819, and BAYR3401.

Non-limiting examples of additional analgesic agents useful in the present methods for treating or preventing pain include acetaminophen, an NSAID, an opiate or a tricyclic antidepressant.

In one embodiment, the other analgesic agent is acetaminophen or an NSAID.

In another embodiment, the other analgesic agent is an opiate.

In another embodiment, the other analgesic agent is a tricyclic antidepressant.

Non-limiting examples of NSAIDS useful in the present methods for treating or preventing pain include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; ; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

Non-limiting examples of opiates useful in the present methods for treating or preventing pain include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

Non-limiting examples of tricyclic antidepressants useful in the present methods for treating or preventing pain include amitryptyline, carbamazepine, gabapentin or pregabalin.

The Pyrimidinedione Derivatives may also be useful in combination (administered together or sequentially in any order) with one or more separate anticancer treatments such as radiation therapy, and/or at least one anticancer agent different from the Pyrimidinedione Derivative. The compounds of the present invention can be present in the same dosage unit as the anticancer agent or in separate dosage units.

Another aspect of the present invention is a method of treating one or more diseases associated with a cyclin dependent kinase, comprising administering to a patient in need of such treatment an amount of a first compound, which is an Pyrimidinedione Derivative, or a pharmaceutically acceptable salt, solvate, ester, prodrug or stereoisomer thereof; and an amount of at least one second compound, the second compound being an anticancer agent different from the Pyrimidinedione Derivative, wherein the amounts of the first compound and the second compound result in a therapeutic effect.

Non-limiting examples of additional anticancer agents suitable for use in the present methods for treating cancer include cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide or teniposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methoxtrexate); alkylating agents (such as temozolomide (TEMODAR™ from Schering-Plough Corporation, Kenilworth, N.J.), cyclophosphamide); Farnesyl protein transferase inhibitors (such as, SARASAR™(4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide, or SCH 66336 from Schering-Plough Corporation, Kenilworth, N.J.), tipifarnib (Zamestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.); signal transduction inhibitors (such as, Iressa (from Astra Zeneca Pharmaceuticals, England), Tarceva (EGFR kinase inhibitors), antibodies to EGFR (e.g., C225), GLEEVEC™ (C-abl kinase inhibitor from Novartis Pharmaceuticals, East Hanover, N.J.); interferons such as, for example, intron (from Schering-Plough Corporation), Peg-Intron (from Schering-Plough Corporation); hormonal therapy combinations; aromatase combinations; ara-C, adriamycin, cytoxan, and gemcitabine.

Other useful additional anticancer agents include but are not limited to Uracil mustard, Chlormethine, Ifosfamide, Melphalan, Chlorambucil, Pipobroman, Triethylenemelamine, ara-C, adriamycin, cytoxan, Clofarabine (Clolar® from Genzyme Oncology, Cambridge, Mass.), cladribine (Leustat® from Janssen-Cilag Ltd.), aphidicolon, rituxan (from Genentech/Biogen Idec), sunitinib (Sutent® from Pfizer), dasatinib (or BMS-354825 from Bristol-Myers Squibb), tezacitabine (from Aventis Pharma), Smll, fludarabine (from Trigan Oncology Associates), pentostatin (from BC Cancer Agency), triapine (from Vion Pharmaceuticals), didox (from Bioseeker Group), trimidox (from ALS Therapy Development Foundation), amidox, 3-AP (3-aminopyridine-2-carboxaldehyde thiosemicarbazone), MDL-101,731 ((E)-2′-deoxy-2′-(fluoromethylene)cytidine) and gemcitabine.

Other useful additional anticancer agents include but are not limited to Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaceuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17□-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Oxaliplatin, Aroplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux, Liposomal, Thiotepa, Altretamine, Melphalan, Trastuzumab, Lerozole, Fulvestrant, Exemestane, Fulvestrant, Ifosfomide, Rituximab, C225 and Campath.

If formulated as a fixed dose, such combination products employ the compounds of this invention within the dosage range described herein and the additional anticancer agent(s) or treatment within its dosage range. For example, the CDC2 inhibitor olomucine has been found to act synergistically with known cytotoxic agents in inducing apoptosis (J. Cell Sci., (1995) 108, 2897. Pyrimidinedione Derivatives may also be administered sequentially with known anticancer or cytotoxic agents when a combination formulation is inappropriate. The invention is not limited in the sequence of administration; Pyrimidinedione Derivatives may be administered either prior to or after administration of the known anticancer or cytotoxic agent. For example, the cytotoxic activity of the cyclin-dependent kinase inhibitor flavopiridol is affected by the sequence of administration with anticancer agents. Cancer Research, (1997) 57, 3375. Such techniques are within the skills of persons skilled in the art as well as attending physicians.

Accordingly, in an aspect, this invention includes methods for treating cancer in a patient, comprising administering to the patient an amount of at least one Pyrimidinedione Derivative, or a pharmaceutically acceptable salt, solvate, ester, prodrug or stereoisomer thereof, and one or more other anticancer treatment modalities, wherein the amounts of the Pyrimidinedione Derivative(s)/other treatment modality result in the desired therapeutic effect. In one embodiment, the at least one Pyrimidinedione Derivative and the one or more other treatment modalities act synergistically. In one embodiment, the at least one Pyrimidinedione Derivative and the one or more other treatment modalities act additively.

In one embodiment, the other treatment modality is surgery.

In another embodiment, the other treatment modality is radiation therapy.

In another embodiment, the other treatment modality is biological therapy, such as hormonal therapy or anticancer vaccine therapy.

In one embodiment, the present combination therapies for treating or preventing diabetes comprise administering a Pyrimidinedione Derivative, an antidiabetic agent and/or an antiobesity agent.

In another embodiment, the present combination therapies for treating or preventing diabetes comprise administering a Pyrimidinedione Derivative and an antidiabetic agent.

In another embodiment, the present combination therapies for treating or preventing diabetes comprise administering a Pyrimidinedione Derivative and an anti-obesity agent.

In one embodiment, the present combination therapies for treating or preventing obesity comprise administering a Pyrimidinedione Derivative, an antidiabetic agent and/or an antiobesity agent.

In another embodiment, the present combination therapies for treating or preventing obesity comprise administering a Pyrimidinedione Derivative and an antidiabetic agent.

In another embodiment, the present combination therapies for treating or preventing obesity comprise administering a Pyrimidinedione Derivative and an anti-obesity agent.

In one embodiment, the additional therapeutic agent is a cholesterol biosynthesis inhibitor. In another embodiment, the cholesterol biosynthesis inhibitor is a squalene synthetase inhibitor. In another embodiment, the cholesterol biosynthesis inhibitor is a squalene epoxidase inhibitor. In still another embodiment, the cholesterol biosynthesis inhibitor is an HMG-CoA reductase inhibitor. In another embodiment, the HMG-CoA reductase inhibitor is a statin. In yet another embodiment, the statin is lovastatin, pravastatin, simvastatin or atorvastatin.

In one embodiment, the additional therapeutic agent comprises a cholesterol absorption inhibitor and a cholesterol biosynthesis inhibitor. In another embodiment, the additional therapeutic agent comprises a cholesterol absorption inhibitor and a statin. In another embodiment, the additional therapeutic agent comprises ezetimibe and a statin. In another embodiment, the additional therapeutic agent comprises ezetimibe and simvastatin.

In one embodiment, the present combination therapies for treating or preventing metabolic syndrome comprise administering a Pyrimidinedione Derivative, an antidiabetic agent and/or an antiobesity agent.

In another embodiment, the present combination therapies for treating or preventing metabolic syndrome comprise administering a Pyrimidinedione Derivative and an antidiabetic agent.

In another embodiment, the present combination therapies for treating or preventing metabolic syndrome comprise administering a Pyrimidinedione Derivative and an anti-obesity agent.

In one embodiment, the present combination therapies for treating or preventing a cardiovascular disease comprise administering one or more Pyrimidinedione Derivatives, and an additional agent useful for treating or preventing a cardiovascular disease.

In addition, the Pyrimidinedione Derivatives can also be used in combination with another therapeutic agent with comprises two or more active ingredients. A non-limiting example of such an additional therapeutic agents is VYTORIN® (a combination of simvastatin and ezetimibe).

When administering a combination therapy to a patient in need of such administration, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Pyrimidinedione Derivatives are administered during a time when the additional therapeutic agent(s) exert their prophylactic or therapeutic effect, or vice versa.

In another embodiment, the one or more Pyrimidinedione Derivatives and the additional therapeutic agent(s) are administered in doses commonly employed when such agents are used as monotherapy for treating or preventing a Condition.

In another embodiment, the one or more Pyrimidinedione Derivatives and the additional therapeutic agent(s) are administered in doses lower than the doses commonly employed when such agents are used as monotherapy for treating or preventing a Condition.

In still another embodiment, the one or more Pyrimidinedione Derivatives and the additional therapeutic agent(s) act synergistically and are administered in doses lower than the doses commonly employed when such agents are used as monotherapy for treating or preventing a Condition.

In one embodiment, the one or more Pyrimidinedione Derivatives and the additional therapeutic agent(s) are present in the same composition. In one embodiment, this composition is suitable for oral administration. In another embodiment, this composition is suitable for intravenous administration.

The one or more Pyrimidinedione Derivatives and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of one or more Pyrimidinedione Derivatives and the additional therapeutic agent(s) may inhibit the resistance of a Condition to these agents.

In one embodiment, when the patient is treated for diabetes or a diabetic complication, the additional therapeutic agent is an antidiabetic agent which is not a Pyrimidinedione Derivative. In another embodiment, the additional therapeutic agent is an agent useful for reducing any potential side effect of a Pyrimidinedione Derivative. Such potential side effects include, but are not limited to, nausea, vomiting, headache, fever, lethargy, muscle aches, diarrhea, general pain, and pain at an injection site.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Pyrimidinedione Derivative(s) and the other agent(s) for treating or preventing diseases or conditions listed above can be administered simultaneously or sequentially. This particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Pyrimidinedione Derivatives and the additional therapeutic agent(s) can, when administered as combination therapy, range from about 0.1 to about 2000 mg per day, although variations will necessarily occur depending on the target of therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The Pyrimidinedione Derivatives may also be deliverable transdermally. The transdermal compositions can take the form of creams, lotions, aerosols and/or emulsions and can be included in a transdermal patch of the matrix or reservoir type as are conventional in the art for this purpose.

In one embodiment, a Pyrimidinedione Derivative is administered orally.

In another embodiment, a Pyrimidinedione Derivative is administered intravenously.

In another embodiment, a Pyrimidinedione Derivative is administered intranasally.

In still another embodiment, a Pyrimidinedione Derivative is administered topically.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 150 mg, preferably from about 1 mg to about 75 mg, more preferably from about 1 mg to about 50 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the Pyrimidinedione Derivatives and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of one or more Pyrimidinedione Derivatives and an additional therapeutic agent, the two active components may be co-administered simultaneously or sequentially, or a single pharmaceutical composition comprising one or more Pyrimidinedione Derivatives and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising an effective amount of one or more Pyrimidinedione Derivatives, or a pharmaceutically acceptable salt, solvate, ester, prodrug or stereoisomer thereof, and a pharmaceutically acceptable carrier.

In another aspect the present invention provides a kit comprising an amount of one or more Pyrimidinedione Derivatives, or a pharmaceutically acceptable salt, solvate, ester, prodrug or stereoisomer thereof, and an amount of at least one additional therapeutic agent listed above, wherein the combined amounts are effective for treating or preventing a Condition in a patient.

When the components of a combination therapy regimen are to be administered in more than one composition, they can be provided in a kit comprising a single package containing one or more containers, wherein one container contains one or more Pyrimidinedione Derivatives in a pharmaceutically acceptable carrier, and a second, separate container comprises an additional therapeutic agent in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 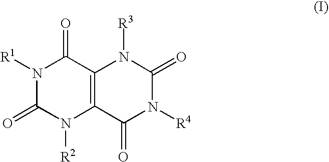
or a pharmaceutically acceptable salt, solvate, ester or prodrug thereof, wherein:
R¹ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, heteroaryl, —OR⁵, —SR⁵, —N(R⁶)₂, —CN, —C(O)OR⁵ and —C(O)N(R⁶)₂;
R² is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵, —NHC(O)—R⁶ and —C(O)N(R⁶)₂;
R³ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R⁴ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
each occurrence of R⁵ is independently H, alkyl, aryl or cycloalkyl;
each occurrence of R⁶ is independently H, alkyl, -(alkylene)ₙ-aryl or cycloalkyl; and
each occurrence of n is independently 0 or 1.

2. The compound of claim 1, wherein R¹ is H or alkyl.

3. The compound of claim 1, wherein R² is H or alkyl.

4. (canceled)

5. The compound of claim 1, wherein R¹ and R² are each alkyl.

6. The compound of claim 1, wherein one of R¹ and R² is H and the other is alkyl.

7. The compound of claim 1, wherein R¹ is methyl or n-pentyl.

8. The compound of claim 1, wherein R² is n-butyl or n-pentyl.

9. The compound of claim 1, wherein R³ and R⁴ are each H.

10-11. (canceled)

12. The compound of claim 5, wherein R³ and R⁴ are, each H.

13. The compound of claim 6, wherein R³ and R⁴ are each H.

14. A compound having the formula: 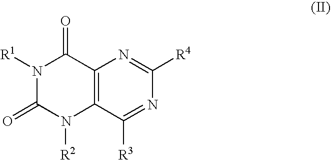
or a pharmaceutically acceptable salt, solvate, ester or prodrug thereof, wherein:
R¹ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R² is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R³ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R⁴ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
each occurrence of R⁵ is independently H, alkyl, aryl or cycloalkyl;
each occurrence of R⁶ is independently H, alkyl, -(alkylene)ₙ-aryl or cycloalkyl; and
each occurrence of n is independently 0 or 1.

15. The compound of claim 14, wherein R¹ is H or alkyl.

16. The compound of claim 14, wherein R² is H or alkyl.

17. (canceled)

18. The compound of claim 14, wherein R¹ and R² are each alkyl.

19. The compound of claim 14, wherein one of R¹ and R² is H and the other is alkyl.

20. The compound of claim 14, wherein R³ is H, alkyl, haloalkyl or —O-alkyl.

21. The compound of claim 14, wherein R⁴ is H, alkyl, haloalkyl or —O-alkyl.

22. The compound of claim 20, wherein R⁴ is H, alkyl, haloalkyl or —O-alkyl.

23. (canceled)

24. A compound having the formula: 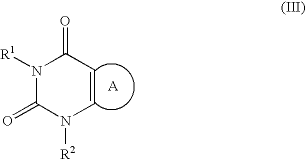
or a pharmaceutically acceptable salt, solvate, ester or prodrug thereof, wherein:
A is: 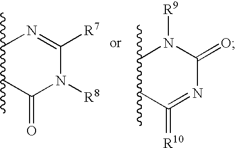
R¹ is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, heteroaryl, —OR⁵, —SR⁵, —N(R⁶)₂, —CN, —C(O)OR⁵ and —C(O)N(R⁶)₂;
R² is H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
each occurrence of R⁵ is independently H, alkyl, aryl or cycloalkyl;
each occurrence of R⁶ is independently H, alkyl, -(alkylene)ₙ-aryl or cycloalkyl;
R⁷ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —O-alkylene-O-aryl, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R⁸ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R⁹ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₂;
R¹⁰ is H, alkyl, haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, cycloalkyl, cycloalkylene, heterocycloalkyl, heterocycloalkenyl or heteroaryl group can be unsubstituted or substituted with up to 4 substituents, which can be the same or different, and are selected from: alkyl, aryl, halo, haloalkyl, —OR⁵, —SR⁵, —N(R⁶)₂, —CN, —C(O)OR⁵ and —C(O)N(R⁶)₇; and
each occurrence of n is independently 0 or 1.

25. The compound of claim 24, wherein A is:

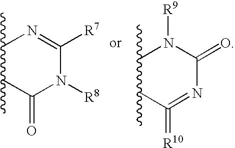

26. (canceled)

27. The compound of claim 25, wherein R¹ and R² are each independently H or alkyl.

28. The compound of claim 27, wherein R¹ and R² are each alkyl.

29. The compound of claim 25, wherein R⁷ is alkyl, —O-alkyl or haloalkyl.

30. The compound of claim 29, wherein R⁷ is —CHF₂.

31. The compound of claim 25, wherein R⁸ is H.

32. The compound of claim 29, wherein R⁸ is H.

33-39. (canceled)

40. A compound having the structure: 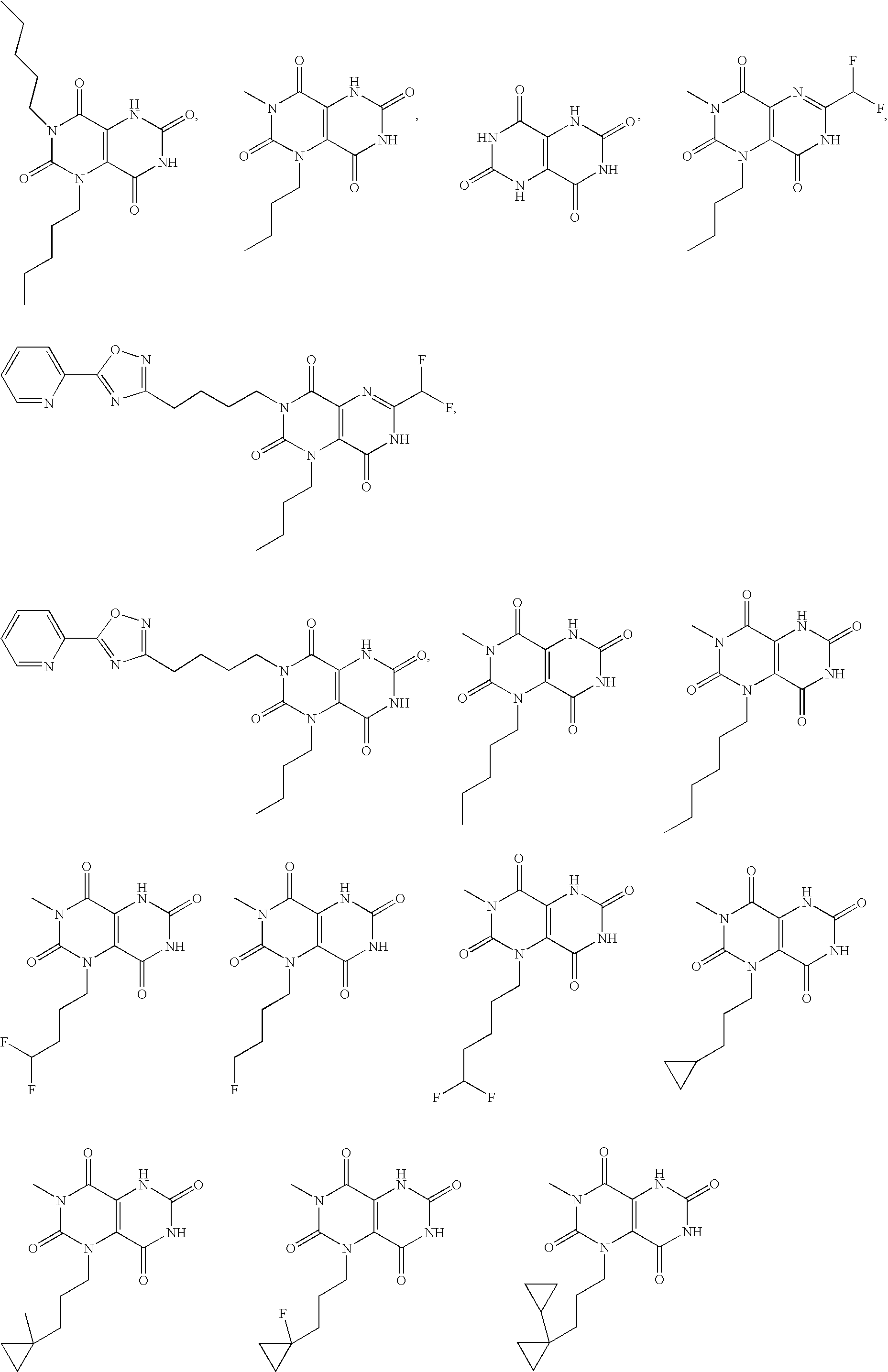 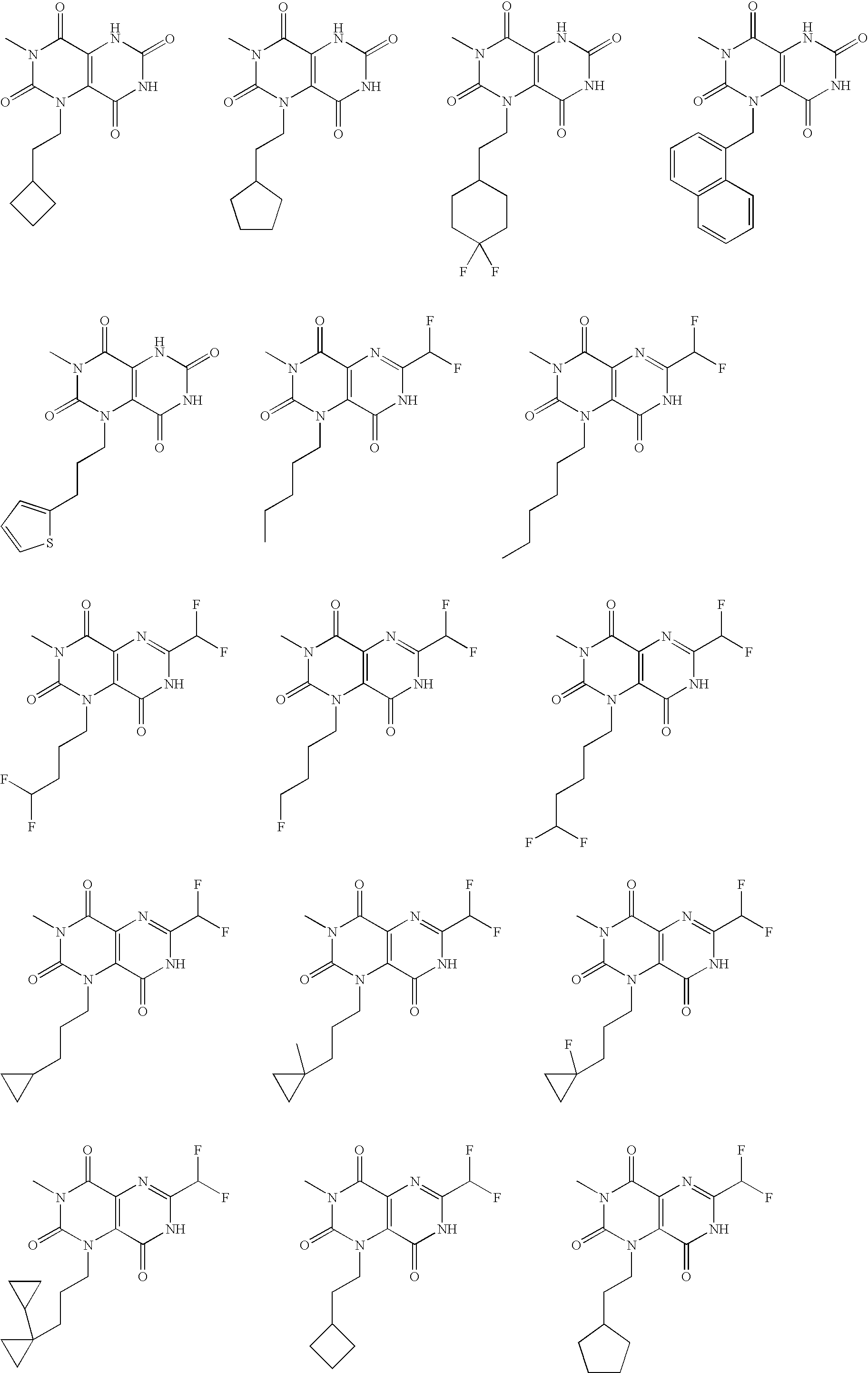 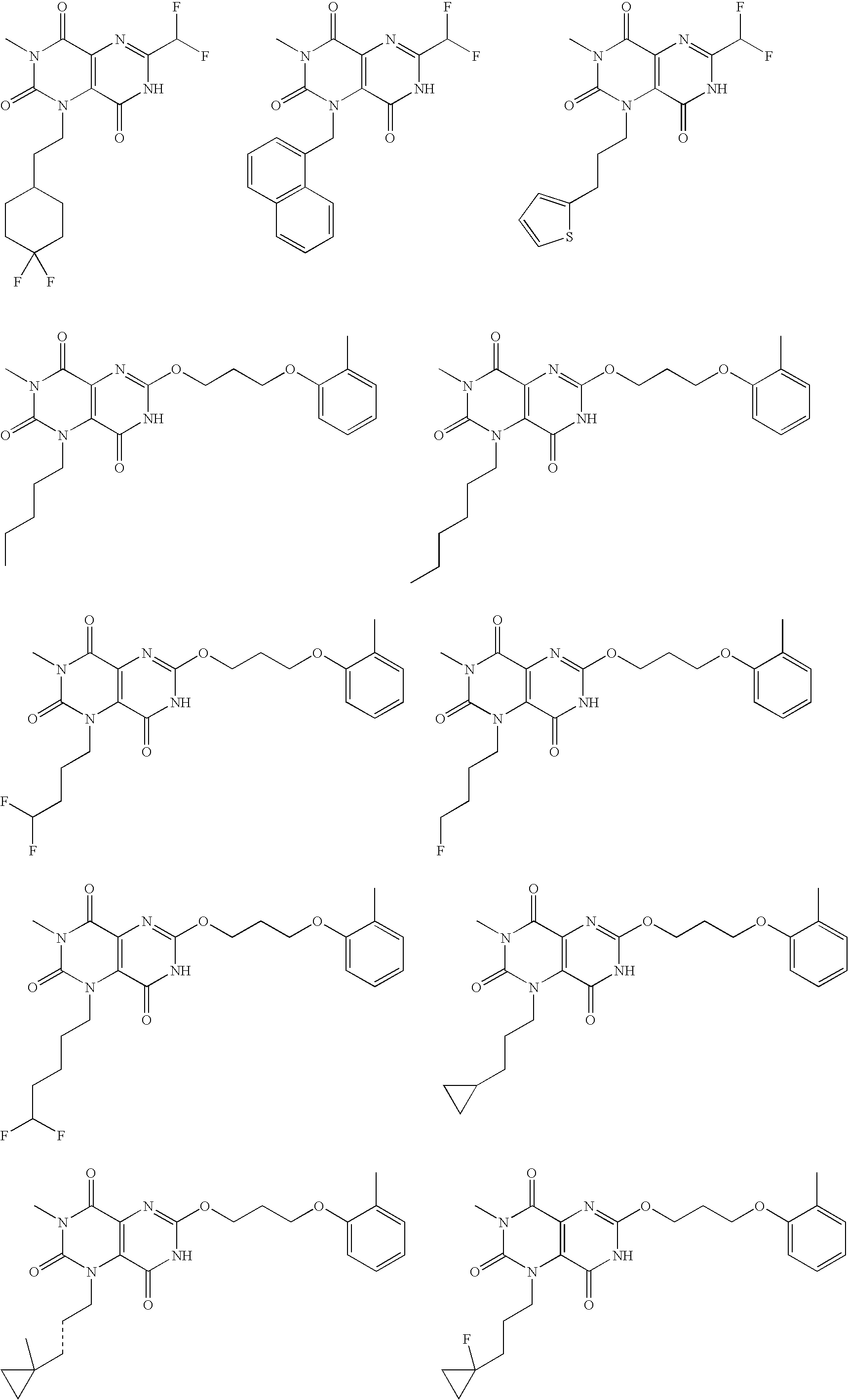 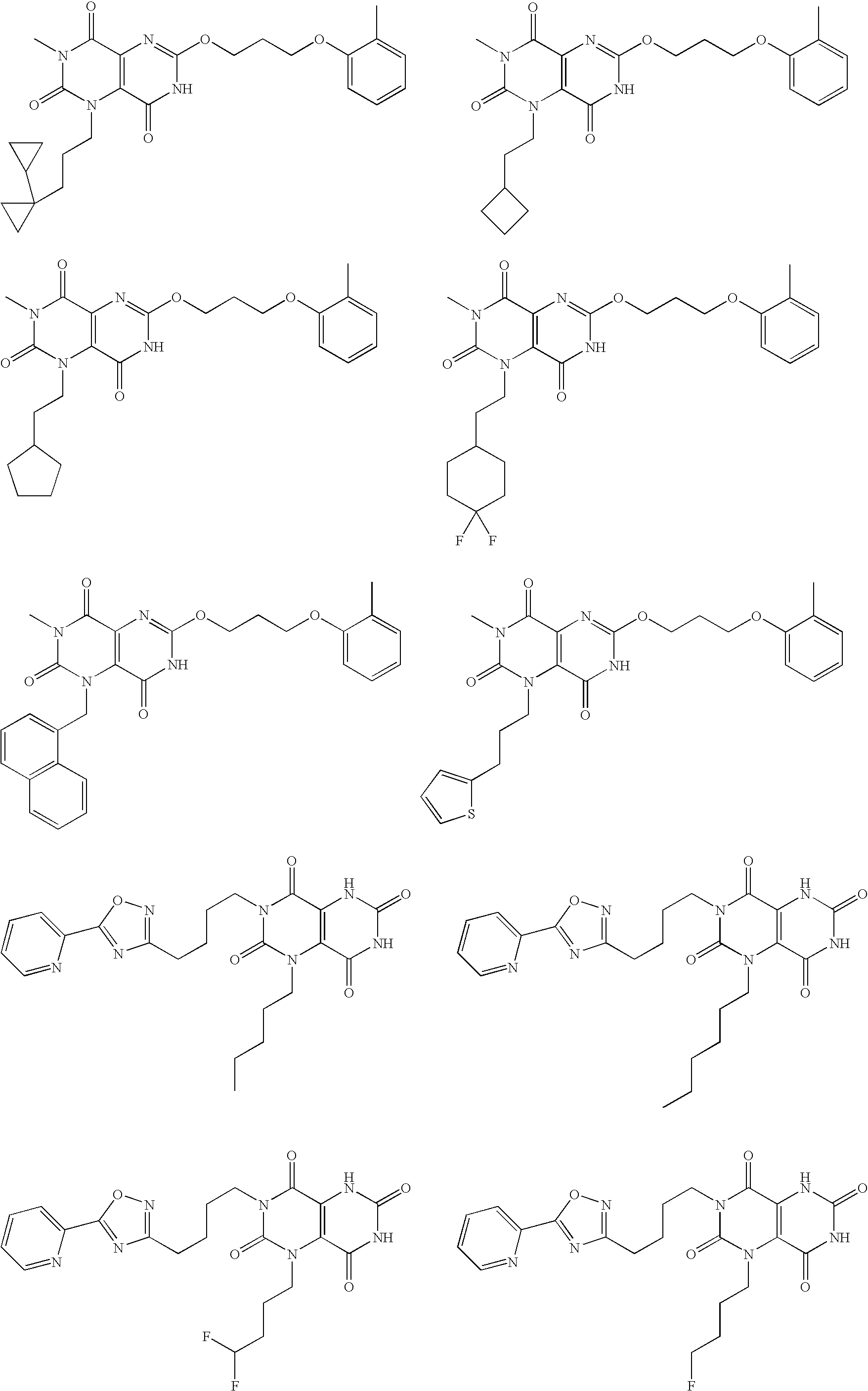 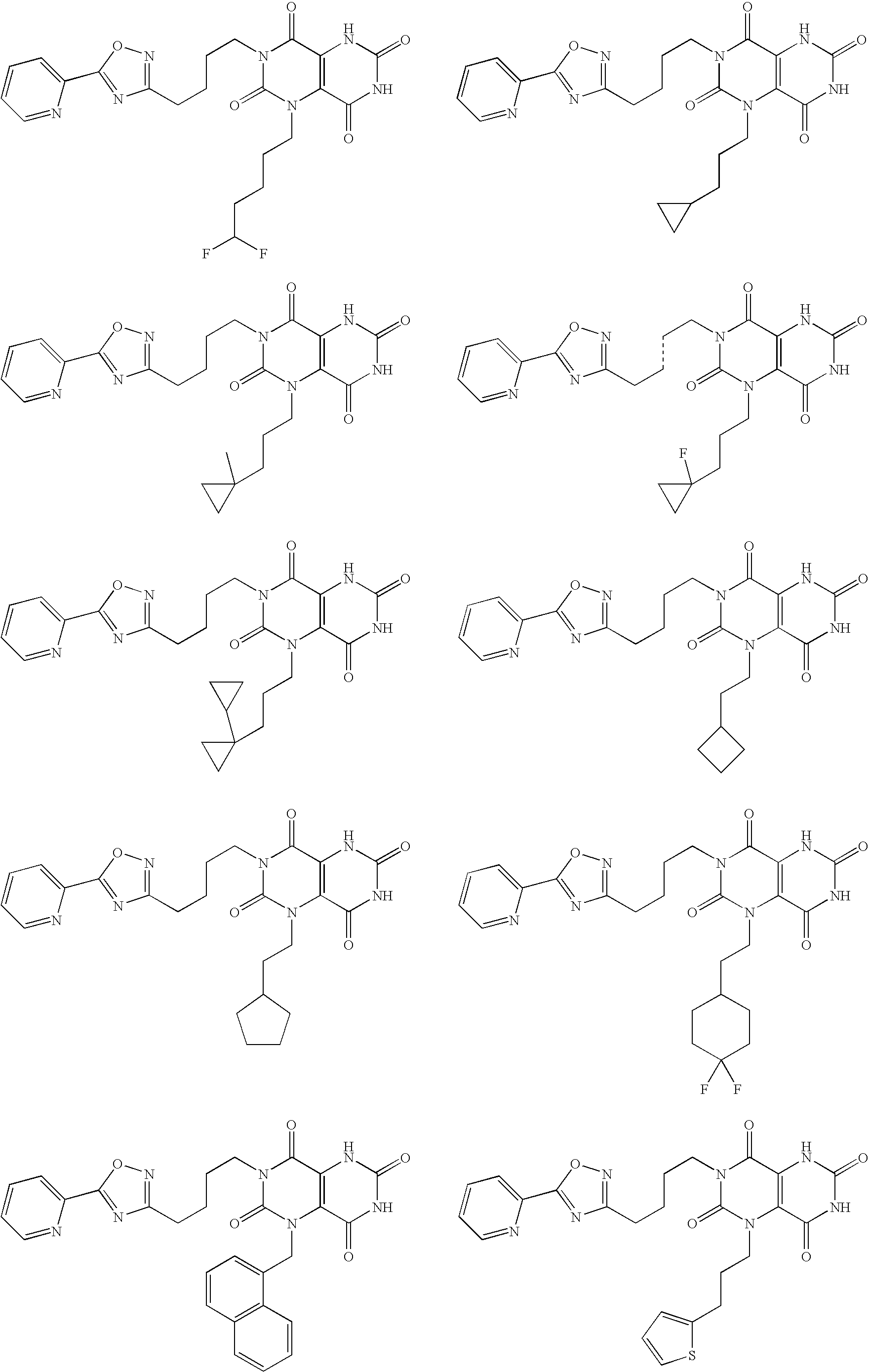 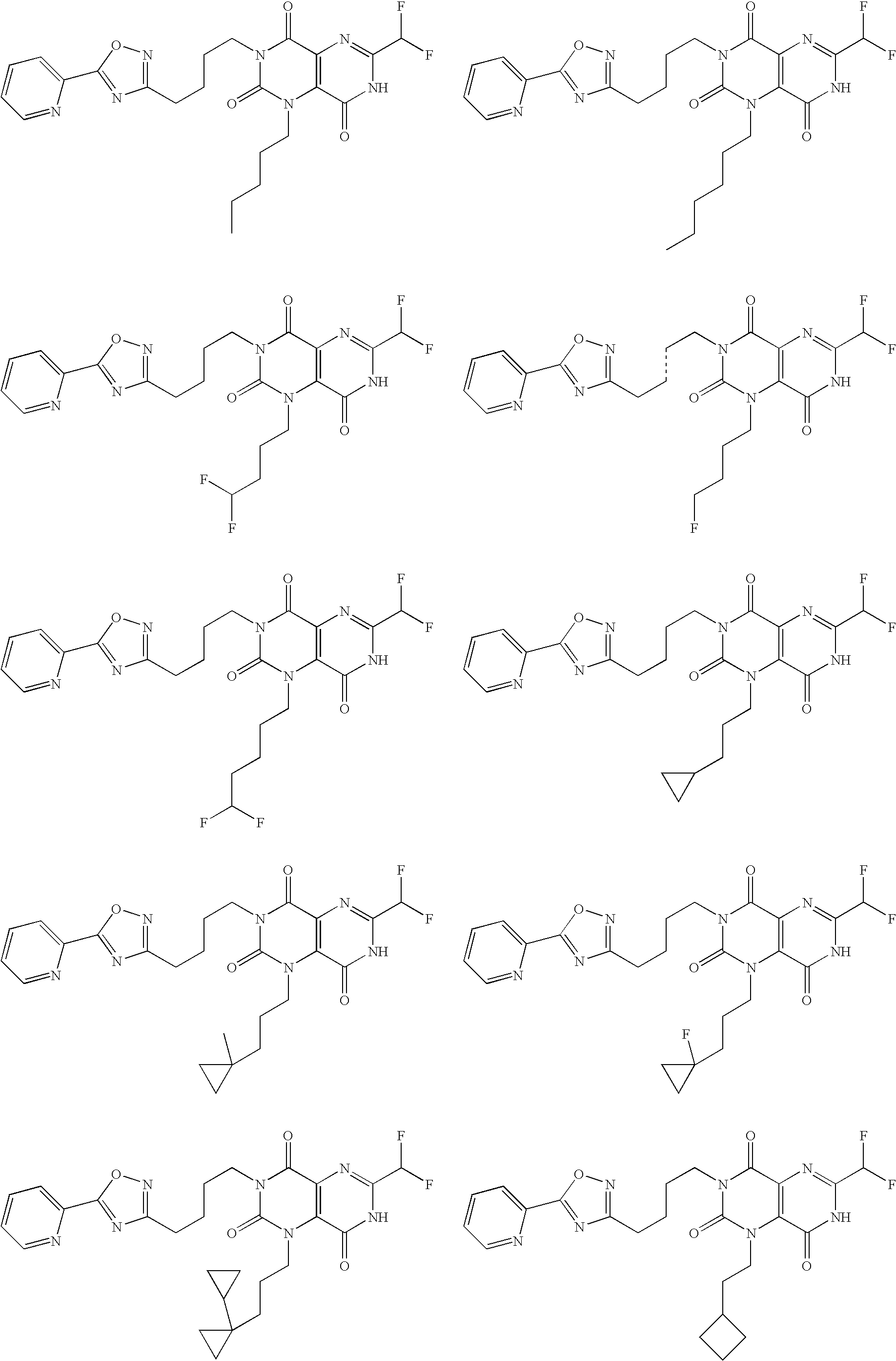 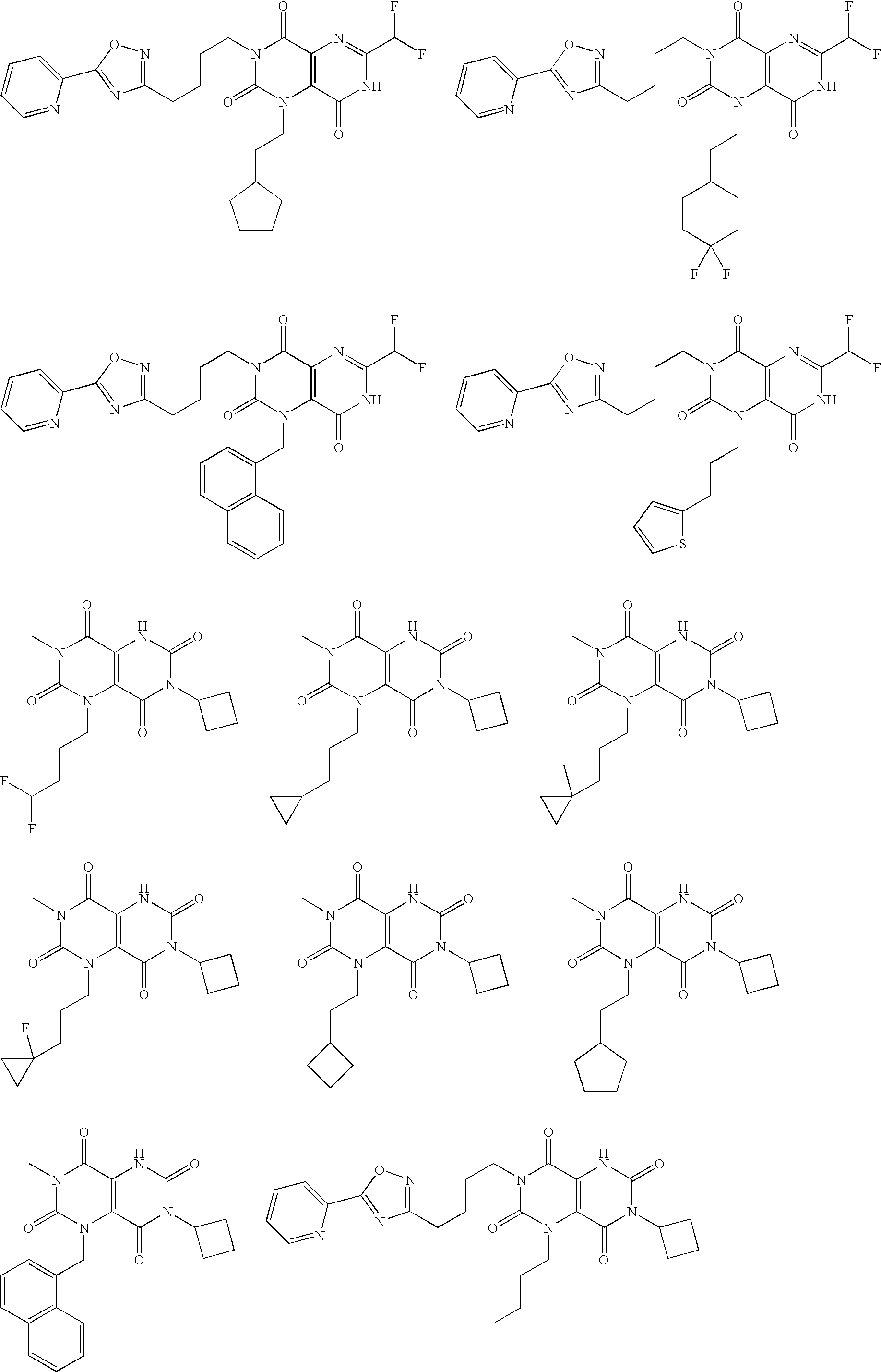 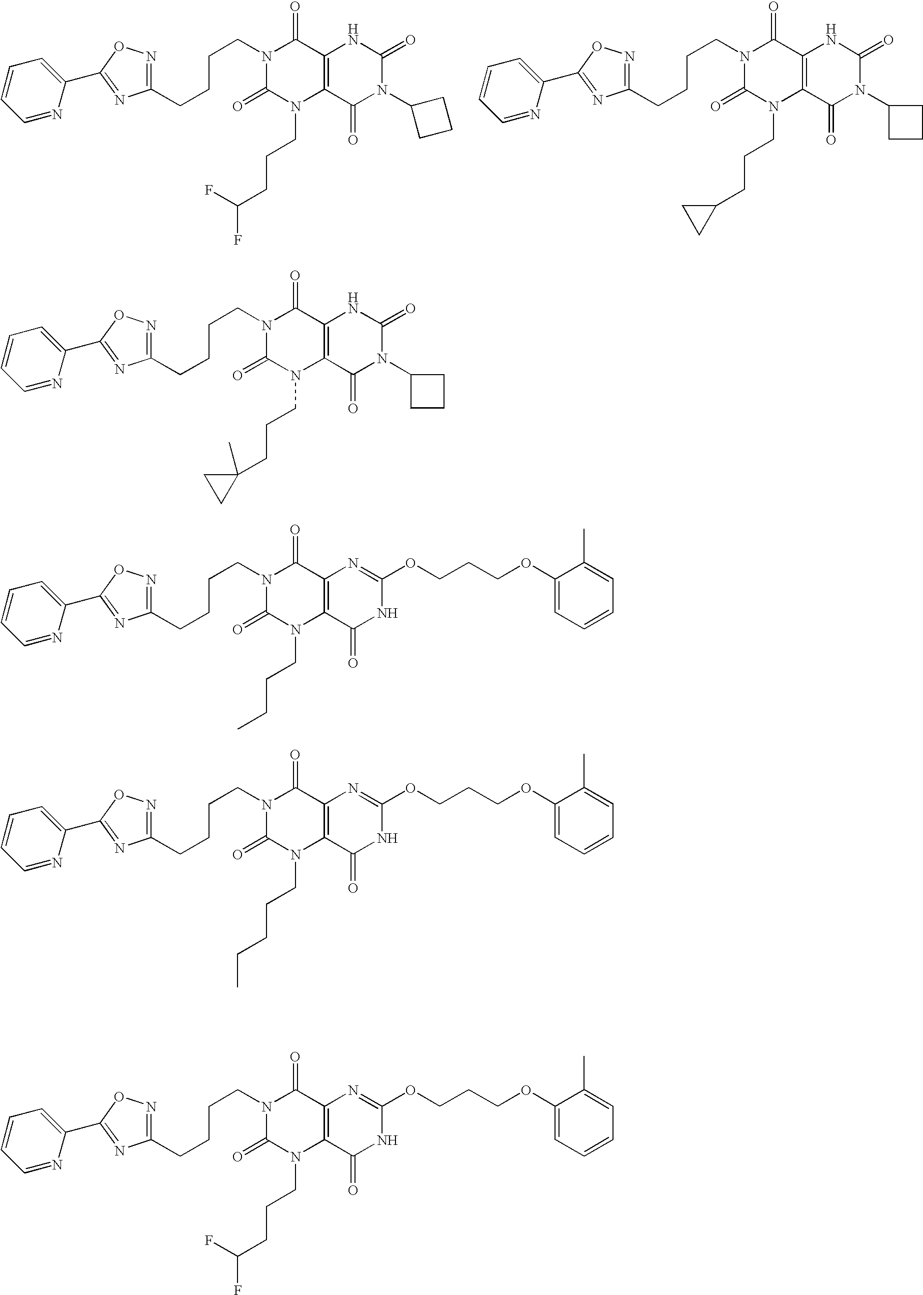 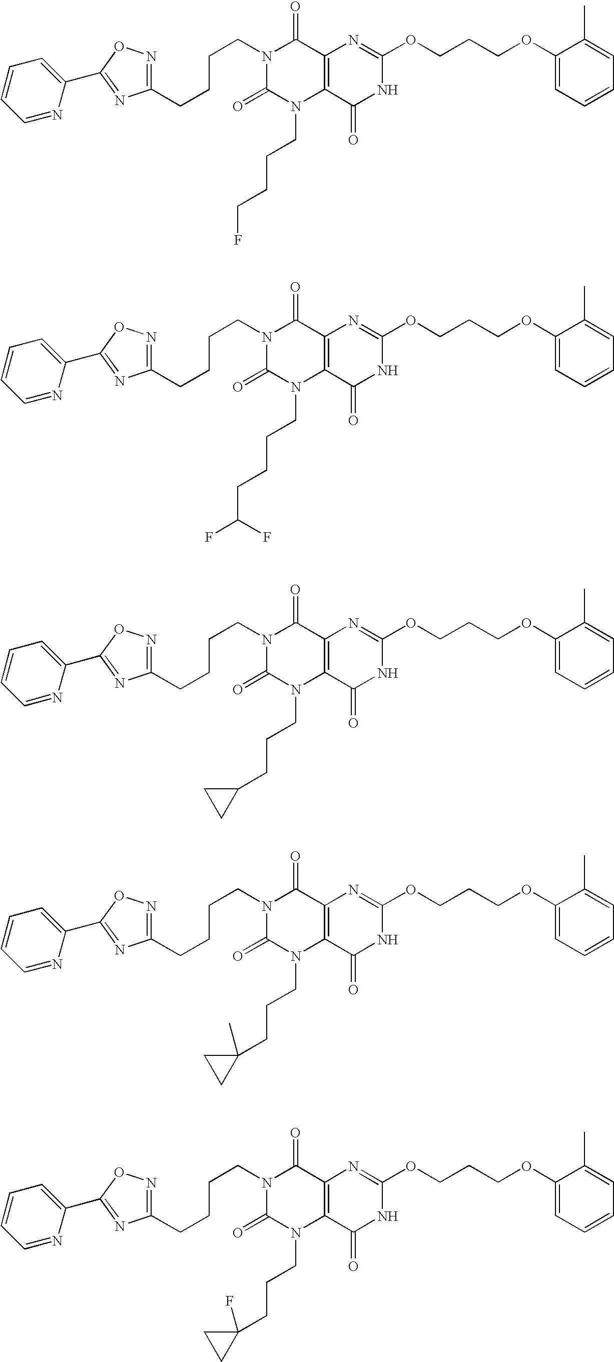 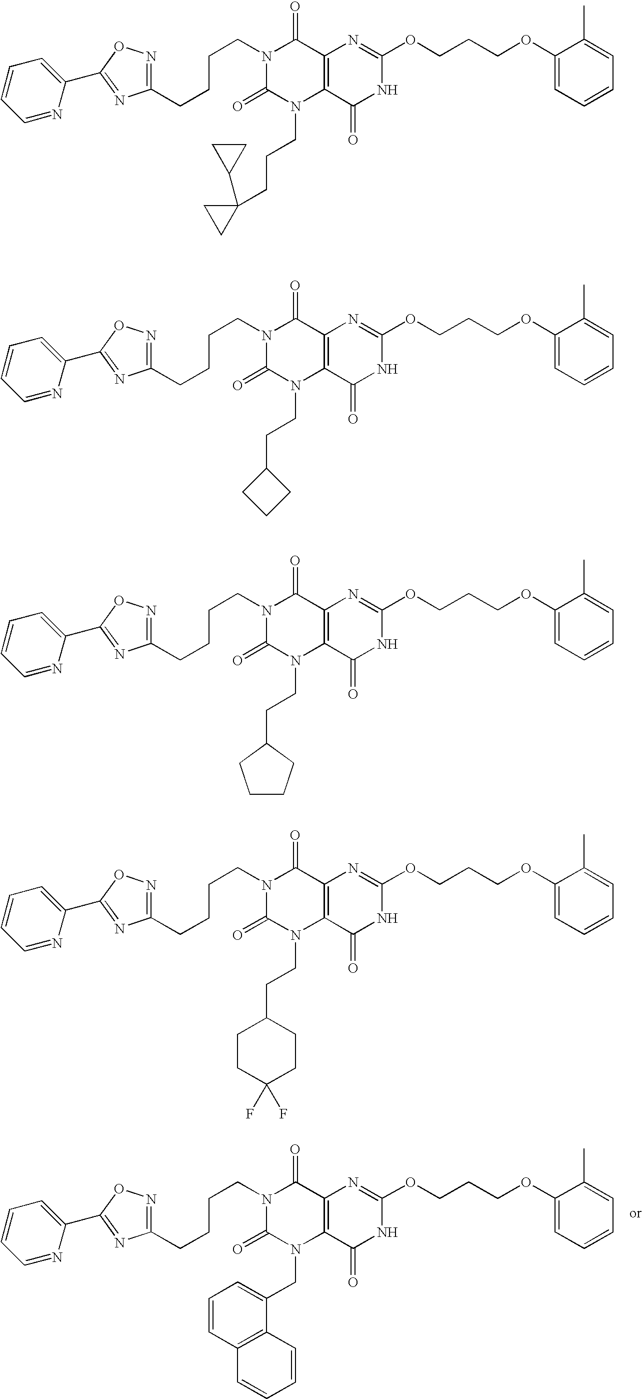 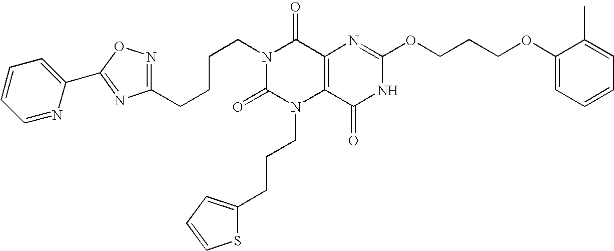
or a pharmaceutically acceptable salt, solvate, ester or prodrug thereof.

41. A composition comprising an effective amount of one or more compounds claim 1 or a pharmaceutically acceptable salt, solvate, ester or prodrug thereof, and a pharmaceutically acceptable carrier.

42. A composition comprising an effective amount of one or more compounds of claim 14 or a pharmaceutically acceptable salt, solvate, ester or prodrug thereof, and a pharmaceutically acceptable carrier.

43. A composition comprising an effective amount of one or more compounds of claim 24 or a pharmaceutically acceptable salt, solvate, ester or prodrug thereof, and a pharmaceutically acceptable carrier.

44. The composition of claim 41, further comprising one or more additional therapeutic agents selected from an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, an ACAT inhibitors, a cholesteryl ester transfer proton protein (CETP) inhibitor and a low-density lipoprotein (LDL) activator.

45. The composition of claim 41, further comprising an HMG-CoA reductase inhibitor selected from the group consisting of lovastatin, simvastatin, pravastatin, atorvastatin, fluvastatin, cerivastatin, rivastatin, rosuvastatin calcium, and pitavastatin.

46-48. (canceled)

49. The composition of claim 42, further comprising one or more additional therapeutic agents selected from an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, ACAT inhibitors, a cholesteryl ester transfer protein (CETP) inhibitor and a low-density lipoprotein (LDL) activator.

50. The composition of claim 42, further comprising an HMG-CoA reductase inhibitor selected from the group consisting of lovastatin, simvastatin, pravastatin, atorvastatin, fluvastatin, cerivastatin, rivastatin, rosuvastatin calcium, and pitavastatin.

51-53. (canceled)

54. The composition of claim 43, further comprising one or more additional therapeutic agents selected from an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, an ACAT inhibitors, a cholesteryl ester transfer protein (CETP) inhibitor and a low-density lipoprotein (LDL) activator.

55. The composition of claim 43, further comprising an HMG-CoA reductase inhibitor selected from the group consisting of lovastatin, simvastatin, pravastatin, atorvastatin, fluvastatin, cerivastatin, rivastatin, rosuvastatin calcium, and pitavastatin.

56-58. (canceled)

59. A method for treating a metabolic disorder, dyslipidemia, a cardiovascular disease, a neurological disorder, a hematological disease, cancer, inflammation, a respiratory disease, a gastroenterological disease, diabetes, a diabetic complication, obesity, an obesity-related disorder or non-alcoholic fatty liver disease in a patient, wherein the method comprises administering to the patient an effective amount of one or compounds of claim 1, or a pharmaceutically acceptable salt, solvate, ester or prodrug thereof.

60. A method for treating a metabolic disorder, dyslipidemia, a cardiovascular disease, a neurological disorder, a hematological disease, cancer, inflammation, a respiratory disease, a gastroenterological disease, diabetes, a diabetic complication, obesity, an obesity-related disorder or non-alcoholic fatty liver disease in a patient, wherein the method comprises administering to the patient an effective amount of one or more compounds of claim 14, or a pharmaceutically acceptable salt, solvate, ester or prodrug thereof.

61. A method for treating a metabolic disorder, dyslipidemia, a cardiovascular disease, a neurological disorder, a hematological disease, cancer, inflammation, a respiratory disease, a gastroenterological disease, diabetes, a diabetic complication, obesity, an obesity-related disorder or non-alcoholic fatty liver disease in a patient, wherein the method comprises administering to the patient an effective amount of one or more compounds of claim 24, or a pharmaceutically acceptable salt, solvate, ester or prodrug thereof.

62. The method of claim 59, further comprising administering to the patient an effective amount of one or more additional therapeutic agents selected from an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, an ACAT inhibitors, a cholesteryl ester transfer protein (CETP) inhibitor and a low-density lipoprotein (LDL) activator.

63. The method of claim 60, further comprising administering to the patient an effective amount of one or more additional therapeutic agents selected from an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, an ACAT inhibitors, a cholesteryl ester transfer protein (CETP) inhibitor and a low-density lipoprotein (LDL) activator.

64. The method of claim 61, further comprising administering to the patient an effective amount of one or more additional therapeutic agents selected from an anti-obesity agent, an antidiabetic agent, an agent useful for treating metabolic syndrome, an agent useful for treating a cardiovascular disease, an agent useful for treating hypercholesterolemia, an agent useful for treating dyslipidemia, a cholesterol biosynthesis inhibitor, a cholesterol absorption inhibitor, a bile acid sequestrant, a probucol derivatives, an IBAT inhibitor, a nicotinic acid derivative, a nicotinic acid receptor (NAR) agonist, an ACAT inhibitors, a cholesteryl ester transfer protein (CETP) inhibitor and a low-density lipoprotein (LDL) activator.
